(19) United States
(12) Patent Application Publication
(10) Pub. No.: US 2023/0115214 A1
(43) Pub. Date: Apr. 13, 2023

(54) DRY POWDER COMPOSITIONS OF OIL-IN-WATER (O/W) EMULSION ADJUVANTED VACCINES

(71) Applicant: BOARD OF REGENTS, THE UNIVERSITY OF TEXAS SYSTEM, Austin, TX (US)

(72) Inventors: Zhengrong CUI, Austin, TX (US); Robert O. WILLIAMS, III, Austin, TX (US); Khaled ABOULFOTOUH, Austin, TX (US); Chaeho MOON, Austin, TX (US); Haiyue XU, Austin, TX (US)

(73) Assignee: BOARD OF REGENTS, THE UNIVERSITY OF TEXAS SYSTEM, Austin, TX (US)

(21) Appl. No.: 17/819,259

(22) Filed: Aug. 11, 2022

Related U.S. Application Data
(60) Provisional application No. 63/232,091, filed on Aug. 11, 2021.

(51) Int. Cl. A61K 39/145; A61K 39/39; A61K 9/19; A61K 47/06; A61K 47/26

(52) U.S. Cl. CPC A61K 39/145; A61K 39/39; A61K 9/19; A61K 47/06; A61K 47/26

(57) ABSTRACT

Described herein is the use of thin film freeze drying methods with oil-in-water adjuvants to produce improved vaccine compositions. This approach solves several major problems associated with the emulsion-adjuvanted vaccines. Additionally, the developed dry powder compositions have the potential to be administered via non-invasive routes (such as intranasal, pulmonary, transcutaneous with or without microneedles) and be stored at ambient temperatures which significantly reduce the costs of vaccination programs.

This application claims the benefit of priority to U.S. Provisional Application No. 63/232,091, filed on Aug. 11, 2021, the entire contents of which are hereby incorporated by reference.

### BACKGROUND

### I. Field

The disclosure generally relates to vaccine compositions. More particularly, the disclosure relates to oil-in-water adjuvants and the preparation of adjuvant/vaccine powders produced from aqueous compositions using thin film freeze drying.

### II. Related Art

Vaccine adjuvants are employed to enhance vaccines' immunogenicity and thus spare the antigens' dose, which could be crucial in the event of a pandemic. They should be adequately formulated for maximum safety and efficacy. Adjuvant formulations include liposomes, virosomes, ISCOMs (i.e., immunostimulatory complex in which protein antigen is incorporated into saponin) and oil-in-water (O/W) emulsions (Alving et al., 2012). Examples of O/W vaccine adjuvants include AS03, MF59® and AddaVax™.

MF59® is an O/W emulsion containing squalene oil (4.3%) in citrate buffer stabilized with non-ionic surfactants (i.e., Tween 80 (0.5%) and Span 85 (0.5%)), having a mean droplet size of 160 nm (Ko & Kang, 2018). AddaVax™ is also a squalene-based O/W nanoemulsion with a similar composition as MF59 (i.e., sorbitan trioleate (0.5% w/v), Tween 80 (0.5% w/v) and squalene oil (5% w/v) in sodium citrate buffer (10 mM, pH 6.5)) and mean droplet size similar to that of MF59®. Both MF59® and AddaVax™ can enhance cellular and humoral immune responses (van Diepen et al., 2018). MF59® is licensed for use in pandemic and seasonal influenza vaccines (e.g., Fluad, Celtura® and Focetria) in many countries (Ko & Kang, 2018).

These vaccines should be stored at 2° C.-8° C. and should not be frozen as accidentally slow freezing causes aggregation or fusion of the O/W emulsion droplets. It was reported that 14%-35% of refrigerators or shipments have exposed vaccines to freezing temperatures which could result in significant loss of freeze-sensitive vaccines (e.g., Fluad®, Celtura® and Focetria®) (Matthias et al., Vaccine. 2007;25(20):3980-6). Not surprisingly, cold chain storage and transport accounts for ˜80% of the cost of vaccination programs in the developing countries (Chen et al., 2011) (AboulFotouh et al., 2021).

As such, improved methods and formulations of O/W emulsion-adjuvanted vaccines that permit less stringent storage conditions and greater overall stability would be highly advantageous.

### SUMMARY

In an aspect is provided a dry vaccine including an antigenic protein and an O/W emulsion adjuvant. In an aspect is provided a pharmaceutical composition including a pharmaceutically acceptable excipient and any of the compositions (e.g., vaccines) described herein (including embodiment). In an aspect is provided a method for preparing a vaccine thin film including applying a liquid vaccine to a freezing surface and allowing the liquid vaccine to disperse and freeze on the freezing surface thereby forming a vaccine thin film. In an aspect is provided a method of treating a disease in a subject in need of such treatment, the method including administering a therapeutically effective amount of a solvated dry vaccine as described herein (e.g., in an aspect, embodiment, example, table, figure, or claims) (e.g., a reconstituted liquid vaccine as described herein) to the patient.

In accordance with the disclosure, there is provided a dry oil-in-water (O/W) emulsion composition, wherein said composition comprises a sugar or a sugar alcohol. The composition may further comprise an antigen, such as an antigen in a subunit vaccine. The sugar or sugar alcohol may be sucrose, trehalose, or mannitol. The composition may have a particle size distribution upon reconstitution within about 10-100% of the range of a corresponding liquid adjuvant composition. The sugar or a sugar alcohol may be present at about 40-90 w/w. The antigen may be influenza virus antigen, diphtheria and tetanus toxoids, hepatitis B surface antigen, major capsid protein of human papilloma virus, and severe acute respiratory syndrome coronavirus 2 (SARS-CoV-2) spike protein, or an antigen to a specific type of tumor. The composition may comprise less than 5% water.

The composition may further comprise an excipient such as a salt, a buffer, a detergent, a polymer, an amino acid, or a preservative. The excipient may be disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. The excipient may be present at 0.3-27.5% w/w.

The composition may be is prepared from a liquid vaccine. The oil component may comprise squalene, such as a composition in which trehalose is present at 40-90% w/w and the oil component is squalene. The composition may further be characterized as an adjuvant composition.

Also provided is a method for preparing an adjuvant thin film comprising applying a liquid adjuvant composition to a freezing surface, wherein said liquid adjuvant composition comprises an oil-in-water (O/W) nanoemulsion and a sugar or sugar alcohol; and allowing said liquid adjuvant composition to disperse and freeze on said freezing surface thereby forming a thin film. The adjuvant thin film may further comprise an antigen, such as an antigen from a subunit vaccine. The sugar or sugar alcohol may be sucrose, trehalose or mannitol. The adjuvant thin film may have a particle size distribution upon reconstitution within 10-100% of the range of the liquid adjuvant composition. The sugar or sugar alcohol may be present at 40-90 w/w. The antigen may be an influenza virus antigen, diphtheria and tetanus toxoids, hepatitis B surface antigen, major capsid protein of human papilloma virus and severe acute respiratory syndrome coronavirus 2 (SARS-CoV-2) spike protein, or an antigen to a specific type of tumor. The adjuvant thin film may comprise less than 5% water.

The adjuvant thin film may further comprise an excipient, such as a salt, a buffer, a detergent, a polymer, an amino acid or a preservative. The excipient may be disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. The excipient may be present at 0.3-27.5% w/w. The oil component may comprise squalene.

The liquid adjuvant composition may be exposed to said freezing surface from about 50 milliseconds to about 5 seconds. Applying may comprise spraying or dripping droplets of said liquid adjuvant composition. The freezing surface temperature may be about −180° C. to 0° C., the diameters of the droplets may be about 2-5 millimeters, and the droplets may be dropped from a distance about 2 cm to 10 cm from the freezing surface. The freezing rate of said droplets may be between 10 K/second and 10³ K/second. The method may further comprise removing the solvent from the liquid adjuvant composition thin film to form a dry adjuvant composition, such as by lyophilization.

The method may further comprise solvating said dry adjuvant composition thereby forming a reconstituted liquid vaccine, such as wherein the vaccine remains immunogenic when administered directly as a powder or upon reconstitution. The solvating of said dry adjuvant composition may be performed at least one year after preparing said dry adjuvant composition from said liquid adjuvant composition. Prior to said solvating of said dry vaccine, the dry vaccine may be stored at about 4° C. for at least 99% of the time. Upon solvating said dry adjuvant composition, the resulting reconstituted liquid adjuvant composition may remain homogeneous for at least one week when stored properly. Upon solvating said dry adjuvant composition, the resulting reconstituted liquid adjuvant composition may not form a precipitate for at least one week when stored properly.

In another embodiment, there is provide a method of inducing an immune response in a subject in need thereof, said method comprising administering a therapeutically effective amount of the dry O/W adjuvant composition as described herein, or a solvated, reconstituted or rehydrated version thereof, to said patient. The O/W adjuvant composition may be administered by inhalation, intranasal administration or transcutaneous administration. The subject may have or be at risk of contracting diphtheria, tetanus, pertussis, influenza, pneumonia, otitis media, bacteremia, meningitis, hepatitis, cirrhosis, anthrax poisoning, rabies, warts, poliomyelitis, Japanese encephalitis, coronavirus diseases, cancer, such as Clostridium tetani, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, coronavirus, or Poliovirus. The subject may have or be at risk of developing a specific type of tumor.

Also provide is a method of inducing an immune response in a subject in need thereof, said method comprising administering a therapeutically effective amount of an adjuvant thin film made according to the methods described herein to a subject. The subject may have or be at risk of contracting diphtheria, tetanus, pertussis, influenza, pneumonia, otitis media, bacteremia, meningitis, hepatitis, cirrhosis, anthrax poisoning, rabies, warts, poliomyelitis, Japanese encephalitis, coronavirus diseases, cancer, such as Clostridium tetani, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, coronavirus, or Poliovirus. The subject may have or be at risk of developing a specific type of tumor.

Additionally, there is provided an adjuvant thin film made according to the methods described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Effect of TFFD process on the droplet size distribution (FIG. 1A) and mean droplet size (FIG. 1B) of AddaVax™ adjuvant. First, the hypothesis that the droplet size distribution of AddaVax™ can be maintained after the liquid O/W emulsion adjuvant is processed into dry powder using TFFD technology and then reconstituted was tested. Two formulations containing either 10 μL (i.e., ADOV-20) or 50 μL (i.e., ADOV-21) AddaVax™ and trehalose (30 mg/mL) were prepared and converted into dry powders using TFFD. Then, the powders were reconstituted, and the droplet size distribution was determined using dynamic light scattering (DLS) with a Malvern Nano Sizer. The formulation compositions are showed in Table 1.

FIGS. 2A-E. Effect of excipient on the mean particle size of AddaVax™-adjuvanted OVA model vaccine. Three formulations (i.e., ADOV-9, ADOV-15 and ADOV-17) were prepared using either trehalose, sucrose or mannitol as an excipient at a concentration of 100 mg/mL. The formulation compositions are showed in Table 1. OVA was dissolved in phosphate buffered saline (PBS, 10 mM, pH 7.2) while the excipients (i.e., trehalose, sucrose or mannitol) were dissolved in a citrate buffer (2.5 mM, pH 6.5).

FIGS. 3A-H. Effect of excipient concentration on the particle size distribution of AddaVax™-adjuvanted OVA model vaccine. Trehalose was employed as the excipient at five concentration levels (i.e., 500, 250, 125, 100 and 50 mg/mL), and sucrose or mannitol were employed at two concentration levels (i.e., 100 and 50 mg/mL). The compositions of these formulations are described in Table 1. Then, all formulations were processed into dry powder using TFFD. The temperature of the cryogenic surface (i.e., the stainless-steel drum of the thin-film freezing device) was kept at −100° C.

FIGS. 4A-C. Effect of conventional shelf freeze-drying on the particle size distribution of AddaVax™-adjuvanted OVA vaccine and SDS-PAGE of freshly prepared as well as TFFD-processed ADOV-7. (FIGS. 4A-B) Two formulations (i.e., ADOV-7 and ADOV-14) having the same composition (Table 1) were prepared. Formulation ADOV-14 (0.5 mL) was subjected to conventional shelf freeze-drying by slowly reducing its temperature form room temperature to −40° C. in a freeze-drier. Formulation ADOV-7 was converted into thin films by dropping onto a drum having a temperature of −100° C. Both formulations were converted into dry powders using the same lyophilization cycle. Then, both powders were reconstituted, and the particle size distribution was determined by DLS. Shown in FIG. 4C is the SDS-PAGE image of OVA as an antigen in ADOV-14 before and after it was subjected to TFFD and reconstitution.

FIG. 5. Effect of buffer concentration (molarity) on the particle size distribution of AddaVax™-adjuvanted OVA model vaccine. Three formulations (ADOV-3, ADOV-7 and ADOV-6) with three different citrate buffer molarities (i.e., 5 mM, 2.5 mM and 1 mM, respectively) were prepared to investigate the effect of buffer molarity on the particle size distribution of the model vaccine. All formulations were processed into dry powders by first forming frozen thin films at drum temperature of −100° C., followed by lyophilization. Then, the TFFD-processed powders were reconstituted, and the particle size distribution was determined using DLS.

FIGS. 6A-D. Effect of antigen type and amount on the particle size distribution of AddaVax™-adjuvanted OVA model vaccine. (FIGS. 6A-B) The effect of antigen amount on the particle size distribution of AddaVax™-adjuvanted OVA model vaccine was investigated at three antigen levels (i.e., 6, 12 and 50 μg/100 μL of the liquid vaccine). Three liquid formulations were prepared (i.e., ADOV-7, ADOV-10 and ADOV-11) and processed into dry powders by first forming frozen thin films at drum temperature of -100° C. followed by lyophilization. Then, the TFFD-processed powders were reconstituted, and the particle size distribution was determined by DLS. (FIGS. 6C-D) A comparison of the representative particle size distribution of AddaVax™ suspension (FIG. 6D) and a model vaccine (i.e., ADOV-19), prepared with lysozyme as an antigen and AddaVax™, subjected to thin-film freeze-drying and reconstitution.

FIG. 7. Effect of processing temperature (i.e., thin-film freezing temperature) on the particle size distribution of the AddaVax™-adjuvanted OVA model vaccine. One formulation composition was prepared and frozen into thin films at different temperatures of the cryogenic surface (i.e., the drum) including −50° C. (i.e., ADOV-12), −100° C. (i.e., ADOV-7) and −180° C. (i.e., ADOV-13). Then, the formed thin films were processed into dry powders by lyophilization. The TFFD-processed powders were reconstituted, and the particle size distribution was determined by DLS.

FIG. 8. Differential scanning calorimetry (DSC) thermogram of TFFD-processed ADOV-7. The powder's glass transition temperature (T_{g}) was determined by DSC to be around 120° C. The moisture content in the powder was determined to be 4.9±0.3% using Karl Fischer Titration.

FIGS. 9A-9E show the in vitro characterization of Fluad Quadrivalent dry powder prepared using TEND. Fluad Quadrivalent contains MF59. (FIG. 9A) mean particle size, (FIG. 9B) PDI and (FIG. 9C) zeta potential values of liquid vaccine (i.e., before TFFD) and the vaccine reconstituted from the dry powder (i.e., after TFFD) as determined using DLS (n=3). (FIG. 9D) SDS-PAGE image. (FIG. 9E) Hemagglutination titers in HAUs/50 μL. The hemagglutination study was repeated twice (n=2) with the same results. Samples for SDS-PAGE analysis and hemagglutination assay were reconstituted in 50 μL water so that the HA content was 6 μg/50 μL to facilitate the analysis. *p<0.05, **p<0.01, ns: non-significant (p<0.05).

FIGS. 10A-10C shows representative particle size distribution curves of AddaVax/OVA vaccine during (FIG. 10A) the formation phase, (FIG. 10B) the fully-developed phase and (FIG. C) the dissipation phase. The powder particle size distribution was determined using Malvern Spraytec laser diffraction system (Malvern Ltd., Malvern, UK) at 6 cm from the tip of a dry powder blower.

### DETAILED DESCRIPTION

Here, the inventors disclose dry powder compositions of AddaVax™, AddaVax™-adjuvanted vaccines and an MF59-adjuvanted human vaccine (Fluad Quadrivalent) as examples of vaccines adjuvanted with O/W emulsions. Formulations containing various excipients at various concentration levels were converted to dry powders using thin-film freeze-drying (TFFD) technology. AddaVax™ adjuvant alone, AddaVax™-adjuvanted vaccines (i.e., ovalbumin (OVA) and lysozyme model vaccines), or MF59-adjuvanted vaccine were dropped onto a cryogenically cooled surface having a temperature between about −20° C. and −180° C. to form frozen thin-films rapidly. The frozen films were then subjected to sublimation to remove water. The dry powders conserved a certain degree of droplet size distribution of the AddaVax™ adjuvant, AddaVax™-adjuvanted model vaccines formulated with various antigens and excipients at different levels, and an MF59-adjuvanted vaccine after TFFD and reconstitution. The dry powders also maintained the function of the antigen in the vaccine.

Generally, dry powders are more easily stored and transported compared to liquid form of the vaccines. They also have the potential to be stored at ambient temperatures or in a controlled temperature chain, which will significantly reduce the costs of the vaccination programs via reducing the costs of cold chain. TFFD is an ultra-rapid freezing process (i.e., 100-1000 K/s) that can preserve, or has only limited effect on, the particle size distribution and immunogenicity of nanoemulsion-adjuvanted vaccines via accelerating the nucleation rate and the formation of small ice crystals. Thus, the use of TFFD methods with O/W adjuvant invention solves several major problems associated with the emulsion-adjuvanted vaccines. Additionally, the developed dry powder compositions have the potential to be administered via non-invasive routes (such as intranasal, pulmonary, transcutaneous with or without microneedles) and be stored at ambient temperatures which significantly reduce the costs of vaccination programs. Of course, the dry powder compositions can be reconstituted for administration by injection.

### I. Definitions

The abbreviations used herein have their conventional meaning within the chemical and biological arts. Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

The terms “a” or “an,” as used in herein means one or more. In addition, the phrase “substituted with a[n],” as used herein, means the specified group may be substituted with one or more of any or all of the named substituents.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, neuropsychiatric exams, and/or a psychiatric evaluation. For example, the certain methods presented herein successfully treat a disease associated with (e.g., caused by) an infectious agent (e.g., bacterium or virus). The term “treating” and conjugations thereof, include prevention of an injury, pathology, condition, or disease. The term “preventing” or “prevention” refers to any indicia of success in protecting a subject or patient (e.g., a subject or patient at risk of developing a disease or condition) from developing, contracting, or having a disease or condition (e.g., an infectious disease or diseases associated with an infectious agent), including preventing one or more symptoms of a disease or condition or diminishing the occurrence, severity, or duration of any symptoms of a disease or condition following administration of a prophylactic or preventative composition as described herein.

An “effective amount” is an amount sufficient for a composition (e.g., compound, vaccine, drug) to accomplish a stated purpose relative to the absence of the composition (e.g., compound, vaccine, drug) (e.g., achieve the effect for which it is administered, treat a disease (e.g., reverse or prevent or reduce severity), reduce spread of an infectious disease or agent, reduce one or more symptoms of a disease or condition, or induce specific antibody or cellular immune responses in the case of vaccines). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a composition (vaccine) is an amount of a composition that, when administered to a subject, will have the intended prophylactic effect, e.g., preventing or delaying the onset (or reoccurrence) of an injury, disease (e.g., infectious disease), pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses (e.g., prime-boost). Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of infection or one or more symptoms of infection in the absence of a composition (e.g., vaccine) as described herein (including embodiments).

“Contacting” is used in accordance with its plain ordinary meaning and refers to the process of allowing at least two distinct species (e.g., compositions, vaccines, bacterium, virus, biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a composition (e.g., vaccine) as described herein and a cell, virus, virus particle, protein, enzyme, or patient. In some embodiments contacting includes allowing a composition described herein to interact with a protein or enzyme that is involved in a signaling pathway. In some embodiments contacting includes allowing a composition described herein to interact with a component of a subject's immune system involved in developing immunity to a component of the composition.

As defined herein, the term “inhibition”, “inhibit”, “inhibiting” and the like in reference to a protein-inhibitor or interaction means negatively affecting (e.g., decreasing) the activity or function of the protein. In some embodiments, inhibition refers to reduction of a disease or symptoms of disease. In some embodiments, inhibition refers to reduction of the growth, proliferation, or spread of an infectious agent (e.g., bacterium or virus). In some embodiments, inhibition refers to preventing the infection of a subject by an infectious agent (e.g., bacterium or virus). In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating the signaling pathway or enzymatic activity or the amount of a protein.

The term “modulator” refers to a composition that increases or decreases the level of a target (e.g., molecule, cell, bacterium, virus particle, protein) or the function of a target or the physical state of the target.

The term “modulate” is used in accordance with its plain ordinary meaning and refers to the act of changing or varying one or more properties. “Modulation” refers to the process of changing or varying one or more properties. For example, as applied to the effects of a modulator on a target, to modulate means to change by increasing or decreasing a property or function of the target or the amount of the target.

“Patient” or “subject in need thereof” refers to a living organism suffering from or prone to a disease or condition that can be treated by administration of a composition (e.g., vaccine or pharmaceutical composition) as provided herein. Non-limiting examples include humans, other mammals, bovines, rats, mice, dogs, monkeys, goat, sheep, cows, deer, and other non-mammalian animals. In some embodiments, a patient is human In some embodiments, a patient or subject in need thereof, refers to a living organism (e.g., human) at risk of developing, contracting, or having a disease or condition associated with an infectious agent (e.g., bacterium or virus).

“Disease” or “condition” refer to a state of being or health status of a patient or subject capable of being treated with the compositions (e.g., vaccines) or methods provided herein. In some embodiments, the disease is a disease related to (e.g., caused by) an infectious agent (e.g., bacterium or virus).

“Pharmaceutically acceptable excipient” and “pharmaceutically acceptable carrier” refer to a substance that aids the administration of an active agent to or absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention. In embodiments, an excipient is a salt, buffer, detergent, polymer, amino acid, or preservative. In embodiments, the excipient is disodium edetate, sodium chloride, sodium citrate, sodium succinate, sodium hydroxide, Sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

The term “preparation” is intended to include the formulation of the active compound with encapsulating material as a carrier providing a capsule in which the active component with or without other carriers, is surrounded by a carrier, which is thus in association with it.

As used herein, the term “administering” means oral administration, administration as a suppository, topical contact, intravenous, intraperitoneal, intramuscular, intralesional, intrathecal, intranasal, intradermal, mucosal, intrarectal, intravaginal, topical, transcutaneous, or subcutaneous administration. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example infection therapies such as antiviral drugs or a vaccine (e.g., different vaccine). The compositions (e.g., vaccines) of the invention can be administered alone or can be coadministered to the patient. Coadministration is meant to include simultaneous or sequential administration of the compounds individually or in combination (more than one composition) and includes vaccine administration in a prime-boost method. Thus, the preparations can also be combined, when desired, with other active substances (e.g., to reduce metabolic degradation, increase immune response (e.g., adjuvant)). The compositions of the present invention can be delivered by transdermally, by a topical route, transcutaneously, formulated as solutions, suspensions, emulsions, gels, creams, ointments, pastes, jellies, paints, powders, and aerosols.

The term “administer (or administering) a vaccine” means administering a composition that prevents or treats an infection in a subject. Administration may include, without being limited by mechanism, allowing sufficient time for the vaccine to induce an immune response in the subject or to reduce one or more symptoms of a disease.

The terms “peptide,” “polypeptide,” and “protein” are used interchangeably herein to refer to a polymer of amino acid residues. An “antigenic protein” is a protein that may be included in a vaccine as an antigen. In embodiments, an antigenic protein may be an antigenic protein conjugated to a sugar (i.e., saccharide) (e.g., monosaccharide, disaccharide, polysaccharide) “antigenic protein saccharide conjugate”. In embodiments, an antigenic protein may be an antigenic protein that is not conjugated to a sugar (saccharide). In embodiments, an antigen protein may be a virus-like particle (VLP). In embodiments, an antigen may be a hapten conjugated to a carrier such as a carrier protein.

The term “peptidyl” and “peptidyl moiety” means a monovalent peptide.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

The terms “dose” and “dosage” are used interchangeably herein. A dose refers to the amount of active ingredient given to an individual at each administration. For the present methods and compositions provided herein, the dose may generally refer to the amount of disease treatment. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition and depends on the route of administration. For example, a dosage form can be in a liquid form for nebulization, e.g., for inhalants, in a tablet or liquid, e.g., for oral delivery, or a saline solution, e.g., for injection.

The term “adjuvant” is used in accordance with its plain ordinary meaning within Immunology and refers to a substance that is commonly used as a component of a vaccine. Adjuvants may increase an antigen specific immune response in a subject when administered to the subject with one or more specific antigens as part of a vaccine. In some embodiments, an adjuvant accelerates an immune response to an antigen. In some embodiments, an adjuvant prolongs an immune response to an antigen. In some embodiments, an adjuvant enhances an immune response to an antigen. In some embodiments, an adjuvant is a squalene-based adjuvant.

The terms “bind”, “bound”, “binding”, and other verb forms thereof are used in accordance with their plain ordinary meaning within Enzymology and Biochemistry and refer to the formation of one or more interactions or contacts between two compositions that may optionally interact. Binding may be intermolecular or intramolecular.

The term “vaccine” is used according to its plain ordinary meaning within medicine and Immunology and refers to a composition including an antigenic component (e.g., antigenic protein) for administration to a subject (e.g., human), which elicits an immune response to the antigenic component (e.g., antigenic protein). In some embodiments a vaccine is a therapeutic. In some embodiments, a vaccine is prophylactic. In some embodiments a vaccine includes one or more adjuvants (e.g., squalene-based adjuvant). A liquid vaccine is a vaccine in liquid form, which may be for example a solution, suspension, emulsion, or dispersion or the antigenic component (e.g., antigenic protein) of the vaccine and may optionally include other components. A dry vaccine is a vaccine comprising 5% or less of water.

A vaccine is a preparation employed to improve immunity to a particular disease. Vaccines include an agent, which is used to induce a response from the immune system of the subject. Various agents that are typically used in a vaccine include, but are not limited to: killed, but previously virulent, micro-organisms; live, attenuated microorganisms; inactivated toxic compounds that are produced by microorganism that cause an illness; protein subunits of microorganisms; conjugates; and nucleic acid-based vaccines such as plasmid DNA vaccines and messenger RNA vaccines. Examples of vaccines that may be converted into a powder vaccine according to the methods described herein include, but are not limited to: influenza vaccine, cholera vaccine, bubonic plague vaccine, polio vaccine, Hepatitis A vaccine, rabies vaccine, yellow fever vaccine, measles vaccine, rubella vaccine, mumps vaccine, typhoid vaccine, tuberculosis vaccine, tetanus vaccine, diphtheria vaccine, diphtheria-tetanus-pertussis vaccine, Hepatitis B vaccine, human papillomavirus (HPV) vaccine, Pneumococcal conjugate vaccines, influenza vaccine, botulism vaccine, polio vaccine, anthrax vaccines, Coronavirus vaccines, and tumor vaccines.

The term “prime-boost” or “prime boost” as applied to a methodology of administering vaccines is used according to its plain ordinary meaning in Virology and Immunology and refers to a method of vaccine administration in which a first dose of a vaccine or vaccine component is administered to a subject or patient to begin the administration (prime) and at a later time (e.g., hours, days, weeks, months later) a second vaccine is administered to the same patient or subject (boost). The first and second vaccines may be the same or different but are intended to both elicit an immune response useful in treating or preventing the same disease or condition. In some embodiments the prime is one or more viral proteins or portions thereof and the boost is one or more viral proteins or portions thereof.

The term “associated” or “associated with” as used herein to describe a disease (e.g., a virus associated disease or bacteria associated disease) means that the disease is caused by, or a symptom of the disease is caused by, what is described as disease associated or what is described as associated with the disease. As used herein, what is described as being associated with a disease, if a causative agent, could be a target for treatment of the disease.

The term “vaccinate”, or additional verb forms thereof, refers to administering a vaccine to a subject (e.g., human) and eliciting an antigen specific immune response, wherein the antigen (e.g., antigenic protein) is included in the vaccine. The term “vaccinate” may also refer to eliciting an antigen specific immune response against an administered antigen (e.g., antigenic protein). In some embodiments, vaccinate is to provide prophylaxis against a disease or infectious agent.

The term “portion” refers to a subset of a whole, which may also be the whole. In some embodiments, a portion is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In some embodiments, a portion is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. Unless indicated otherwise, the term “about” in the context of a numeric value indicates the nominal value ±10% of the nominal value. In some embodiments, “about” may be the nominal value.

### II. Compositions

In one aspect, there is provided a dry vaccine including an oil-in-water (O/W) adjuvant. An example is AddaVax™, which is available in a liquid form that should be stored between 2° C. and 8° C. It is based on nano-emulsification of two components, sorbitan trioleate (0.5% w/v) in squalene oil (5% v/v) and Tween 80 (0.5% w/v) in sodium citrate buffer (10 mM, pH 6.5) The nano-emulsion is produced using a microfluidizer and filtered through a 0.22-μm filter to remove large droplets and sterilize the final product. The particle size is ˜160 nm.

AddaVax™ is a squalene-based O/W nano-emulsion. Squalene is an oil more readily metabolized than the paraffin oil used in Freund's adjuvants. AddaVax™ promotes a significant increase in antibody titers with reportedly more balanced Th1/Th2 responses than those obtained with alum. AddaVax™ is believed to act through a depot effect, enhancement of antigen persistence at the injection site, recruitment and activation of antigen presenting cells, and direct stimulation of cytokines and chemokines production by macrophages and granulocytes.

Another O/W adjuvant is MF59, an O/W nanoemulsion composed of squalene and two surfactants, Tween 80 and Span 85. It should be stored at 2° C. to 8° C. and should not be frozen. The mechanism of MF59® is believed to involve rapid induction of chemokines, inflammatory cytokines, recruiting multiple immune cells including antigen-presenting cells, and inducing benign apoptosis of certain innate immune cells. While there is some evidence of CD4+ T cell involvement, the adjuvant effects of MF59® on generating vaccine-specific isotype-switched IgG antibodies, effector CD8+ T cells, and protective immunity are retained even in a CD4+ T cell-deficient model.

If O/W-adjuvanted vaccines are unintentionally exposed to freezing during transport or storage, they should be discarded, resulting in significant loss of vaccines. Herein, the inventors address such shortcomings by rapid freezing of such O/W adjuvants alone or O/W adjuvanted-vaccines into frozen thin films that are subsequently lyophilized into dry powders by a method named thin-film freeze-drying (TFFD). The dry powders successfully maintained the particle size distribution of the adjuvant and adjuvanted vaccines after reconstitution before administration. The inventors have shown that the integrity of the antigens in the resultant adjuvanted vaccine powders was maintained after TFFD. Additionally, the developed dry powders have the potential to be stored at ambient temperatures and be administrated via noninvasive routes (e.g., inhalation or intranasal).

Other available technologies can be utilized to convert liquid vaccines to dry powders (e.g., spray-drying and conventional shelf freeze-drying). Conventional shelf freeze-drying is the most utilized powder engineering technology to transform small molecule drugs as well as vaccines and other biologics into dry powders while maintaining stability and sterility (Amorij et al., 2007). However, we have shown that the mean particle size of reconstituted dry powder of AddaVax™-adjuvanted model vaccine prepared by conventional shelf freeze-drying significantly increased (i.e., mean particle size increased by about 100%, with an additional group of larger particles in the particle size distribution curve). The low cooling rate of conventional shelf freeze drying (i.e., 1-10 K/min) likely resulted in phase separation and thus damage of the AddaVax™ nanoemulsion and the protein antigens (e.g., denaturation and/or aggregation) (Heller et al., 1997)(Randolph, 1997). TFFD is invented by one of the co-inventors of the present invention (Williams III) and achieves ultra-rapid cooling (i.e., 100-1000 K/s), which in turn likely preserves particle size distribution and antigen integrity via accelerating the nucleation rate and the formation of small ice crystals.

In embodiments, at least 60% of the antigenic protein is not denatured. In embodiments, at least 70% of the antigenic protein is not denatured. In embodiments, at least 80% of the antigenic protein is not denatured. In embodiments, at least 90% of the antigenic protein is not denatured. In embodiments, at least 95% of the antigenic protein is not denatured. In embodiments, at least 60% of the antigenic protein is in a conformationally native state. In embodiments, at least 70% of the antigenic protein is in a conformationally native state. In embodiments, at least 80% of the antigenic protein is in a conformationally native state. In embodiments, at least 90% of the antigenic protein is in a conformationally native state. In embodiments, at least 95% of the antigenic protein is in a conformationally native state. A “conformationally native state” is a folded conformation corresponding to an operative or functional protein. A “denatured” protein is a protein having a conformation differing from the folded active or functional conformation of the protein, wherein the denatured protein has a reduced level of activity or function. In embodiments, the antigenic protein is an unconjugated antigenic protein. In embodiments, the antigenic protein is an antigenic protein sugar (saccharide) conjugate. In embodiments, the sugar (saccharide) is a monosaccharide. In embodiments, the sugar (saccharide) is a disaccharide. In embodiments, the sugar (saccharide) is a polysaccharide.

In embodiments, the dry vaccine may include less than 5% water. In embodiments, the dry vaccine includes less than 4% water. In embodiments, the dry vaccine includes less than 3% water. In embodiments, the dry vaccine includes less than 2% water. In embodiments, the dry vaccine includes less than 1% water. In embodiments, the dry vaccine includes less than 5% water (wt/wt). In embodiments, the dry vaccine includes less than 4% water (wt/wt). In embodiments, the dry vaccine includes less than 3% water (wt/wt). In embodiments, the dry vaccine includes less than 2% water (wt/wt). In embodiments, the dry vaccine includes less than 1% water (wt/wt). In embodiments, the dry vaccine includes about 5% water. In embodiments, the dry vaccine includes about 4% water. In embodiments, the dry vaccine includes about 3% water. In embodiments, the dry vaccine includes about 2% water. In embodiments, the dry vaccine includes about 1% water. In embodiments, the dry vaccine includes about 5% water (wt/wt). In embodiments, the dry vaccine includes about 4% water (wt/wt). In embodiments, the dry vaccine includes about 3% water (wt/wt). In embodiments, the dry vaccine includes about 2% water (wt/wt). In embodiments, the dry vaccine includes about 1% water (wt/wt). In embodiments, the dry vaccine includes less than 5% water (v/v). In embodiments, the dry vaccine includes less than 4% water (v/v). In embodiments, the dry vaccine includes less than 3% water (v/v). In embodiments, the dry vaccine includes less than 2% water (v/v). In embodiments, the dry vaccine includes less than 1% water (v/v). In embodiments, the dry vaccine includes about 5% water (v/v). In embodiments, the dry vaccine includes about 4% water (v/v). In embodiments, the dry vaccine includes about 3% water (v/v). In embodiments, the dry vaccine includes about 2% water (v/v). In embodiments, the dry vaccine includes about 1% water (v/v).

In embodiments, the dry vaccine includes an excipient. In embodiments, the dry vaccine includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, Sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer In embodiments, the excipient is trehalose. In embodiments, the dry vaccine includes less than 30-98% wt/wt of all excipients, including sugars or sugar alcohols. In embodiments, the dry vaccine includes about 40-95% wt/wt of the sugar, such as trehalose. In embodiments, the dry vaccine includes at or less than about 90% wt/wt of the sugar, such as trehalose. In embodiments, the dry vaccine at or less than about 80% wt/wt of the sugar, such as trehalose. In embodiments, the dry vaccine includes at or less than about 70% wt/wt of the sugar, such as trehalose. In embodiments, the dry vaccine includes at or about 60% wt/wt of the sugar, such as trehalose. In embodiments, the dry vaccine includes at or about 50% wt/wt of the sugar, such as trehalose. In embodiments, the dry vaccine includes at or about 40% wt/wt of the sugar, such as trehalose. In embodiments, the dry vaccine includes at or about 50-80% wt/wt of the sugar, such as trehalose.

In embodiments, the dry vaccine includes particles, wherein the particles include the O/W emulsion droplets. In embodiments, the dry vaccine is prepared from a liquid vaccine.

In an embodiment, a powder (e.g., dry) vaccine, which retains its efficacy, may be made from a vaccine composition. The method includes obtaining a liquid (e.g., aqueous) vaccine composition. The vaccine composition includes an agent that resembles a disease-causing microorganism, or a compound associated with the disease-causing microorganism (e.g., antigenic protein). The vaccine composition also includes an adjuvant (e.g., squalene-based adjuvant). The vaccine composition is frozen to obtain a frozen vaccine composition (e.g., frozen vaccine thin film). Water is removed from the frozen vaccine composition to form a powder (e.g., dry) vaccine that includes the agent or compound (e.g., antigenic protein) and the adjuvant (e.g., squalene-based adjuvant).

A cryoprotectant may be added to the vaccine composition to protect the organisms (either live or dead) or agents present in the composition from damage during the freezing process. Examples of cryoprotectants include monosaccharides and polysaccharides (e.g., trehalose). A cryoprotectant may be present in amounts up to about 40-90% by weight.

Additionally, the solid form of the vaccine is expected to be advantageous over vaccine in liquid dispersion (i.e., suspension) for stockpiling vaccines that are critical to national security and public health. For example, botulism is a life-threatening disease caused by botulinum neurotoxins (BoNTs), which are produced by one of the seven structurally similar Clostridium botulinum serotypes, designated A to G. Each of the toxins is immunologically distinct, except that serotypes C and D share significant cross-homology. BoNTs are the most poisonous substances known in nature. A single gram of crystalline toxin, evenly dispersed and inhaled, would kill more than one million people. Previously, an investigational pentavalent botulism toxoid (PBT) vaccine aiming to protect against BoNT serotypes A-E had been available. However, as of November 2011, the PBT vaccine has been discontinued by the Centers for Disease Control and Prevention (CDC), based on “an assessment of the available data, which indicate a decline in immunogenicity of some of the toxin serotypes”. Since the investigative PBT vaccine was the only botulism vaccine available in the U.S., discontinuation of it has significant national security implications.

In another embodiment, an aqueous vaccine composition may be composed of an agent that forms particles having a particle size of less than about 500 nm (e.g., less than 10, 20, 30, 40, 50, 60, 70, 80, 90, 100, 110, 120, 130, 140, 150, 160, 170, 180, 190, 200, 250, 300, 350, 400, 450, or 500 nm). In some embodiments, particles having a diameter of less than 500 nm (e.g., less than 10, 20, 30, 40, 50, 60, 70, 80, 90, 100, 110, 120, 130, 140, 150, 160, 170, 180, 190, 200, 250, 300, 350, 400, 450, or 500 nm) may be used as adjuvants in a vaccine composition. The aqueous vaccine composition may be used to vaccinate a subject against the disease related to the agent. In some embodiments, the aqueous vaccine composition can be converted to a vaccine powder, as described above, for storage, for use as an inhalant, or use in other delivery modes.

In embodiments, a dry vaccine is the dry vaccine described herein, including in embodiments, examples, tables, figures, and claims. In embodiments, a dry vaccine is a dry vaccine made by a method described herein, including in aspects, embodiments, examples, tables, figures, and claims. Provided herein is a reconstituted liquid vaccine comprising a dry vaccine as described herein (including in an aspect, embodiment, example, table, figure, or claim) or a dry vaccine prepared using a method as described herein (including in an aspect, embodiment, example, table, figure, or claim) and a solvent (e.g., water, buffer, solution, liquid including an excipient).

Provided in another aspect is a pharmaceutical composition including a pharmaceutically acceptable excipient and any of the compositions (e.g., vaccines) described herein (including embodiment).

The compositions described herein (including embodiments and examples) can be administered alone or can be coadministered to the patient. Coadministration is meant to include simultaneous or sequential administration of the compositions individually or in combination (more than one composition). Thus, the preparations can also be combined, when desired, with other active substances (e.g., to reduce metabolic degradation, increase immune response (e.g., adjuvants)). An example of coadministration of vaccine compositions is a prime-boost method of administration.

Pharmaceutical compositions provided by the present invention include compositions wherein the active ingredient (e.g., compositions described herein, including embodiments) is contained in a therapeutically or prophylactically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., prevent infection, and/or reducing, eliminating, or slowing the progression of disease symptoms. Determination of a therapeutically or prophylactically effective amount of a composition of the invention is well within the capabilities of those skilled in the art, especially in light of the detailed disclosure herein.

### II. Methods

In an aspect is provided a method for preparing an O/W emulsion thin film or an O/W adjuvanted vaccine thin film including applying a liquid emulsion or vaccine to a freezing surface; allowing the liquid emulsion or vaccine to disperse and freeze on the freezing surface thereby forming a vaccine thin film.

In embodiments, the liquid vaccine includes an excipient. In embodiments, the liquid vaccine includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, Sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer

In embodiments, the excipient is a sugar, such as trehalose. In embodiments, the liquid vaccine includes about 3 to 50% wt/vol of the sugar/liquid vaccine, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% wt/vol.

In embodiments, the liquid vaccine includes less than 5% wt/vol of the remaining excipients/liquid vaccine, such as equal to or less than 4%, 3%, 2%, 1%, 0.5%, 0.25, or 0.1 wt/vol of the other excipient/liquid vaccine.

In embodiments, the applying includes spraying or dripping droplets of the liquid vaccine. In embodiments, the vapor-liquid interface of the droplets is less than 500 cm⁻¹ area/volume. In embodiments, the vapor-liquid interface of the droplets is less than 400 cm⁻¹ area/volume. In embodiments, the vapor-liquid interface of the droplets is less than 300 cm⁻¹ area/volume. In embodiments, the vapor-liquid interface of the droplets is less than 200 cm⁻¹ area/volume. In embodiments, the vapor-liquid interface of the droplets is less than 100 cm⁻¹ area/volume. In embodiments, the vapor-liquid interface of the droplets is less than 50 cm⁻¹ area/volume. In embodiments, the vapor-liquid interface of the droplets is less than 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹ area/volume.

In embodiments, the method further includes contacting the droplets with a freezing surface having a temperature below the freezing temperature of the liquid vaccine (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 1450, 160, 170 or 180° Celsius below 0). In embodiments, the method further includes contacting the droplets with a freezing surface having a temperature differential of at least 30° C. between the droplets and the surface. In embodiments, the temperature differential is at least 40° C. between the droplets and the surface. In embodiments, the temperature differential is at least 50° C. between the droplets and the surface. In embodiments, the temperature differential is at least 60° C. between the droplets and the surface. In embodiments, the temperature differential is at least 70° C. between the droplets and the surface. In embodiments, the temperature differential is at least 80° C. between the droplets and the surface. In embodiments, the temperature differential is at least 90° C. between the droplets and the surface. In embodiments, the temperature differential between the droplets and the surface is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C.

In embodiments, the vaccine thin film has a thickness of less than 5000 micrometers. In embodiments, the vaccine thin film has a thickness of less than 4000 micrometers. In embodiments, the vaccine thin film has a thickness of less than 3000 micrometers. In embodiments, the vaccine thin film has a thickness of less than 2000 micrometers. In embodiments, the vaccine thin film has a thickness of less than 1000 micrometers. In embodiments, the vaccine thin film has a thickness of less than 500 micrometers. In embodiments, the vaccine thin film has a thickness of less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers. In embodiments, the vaccine thin film has a thickness of about 500 micrometers. In embodiments, the vaccine thin film has a thickness of about 400 micrometers. In embodiments, the vaccine thin film has a thickness of about 300 micrometers. In embodiments, the vaccine thin film has a thickness of about 200 micrometers. In embodiments, the vaccine thin film has a thickness of about 100 micrometers. In embodiments, the vaccine thin film has a thickness of about 50 micrometers. In embodiments, the vaccine thin film has a thickness of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers.

In embodiments, the vaccine thin film has a surface area to volume ratio of between 5 and 500 cm⁻¹. In embodiments, the vaccine thin film has a surface area to volume ratio of between 5 and 400 cm⁻¹. In embodiments, the vaccine thin film has a surface area to volume ratio of between 5 and 300 cm⁻¹. In embodiments, the vaccine thin film has a surface area to volume ratio of between 5 and 200 cm⁻¹. In embodiments, the vaccine thin film has a surface area to volume ratio of between 5 and 100 cm⁻¹. In embodiments, the vaccine thin film has a surface area to volume ratio of between 5 and 50 cm⁻¹. In embodiments, the vaccine thin film has a surface area to volume ratio of between 5 and 40 cm⁻¹. In embodiments, the vaccine thin film has a surface area to volume ratio of between 5 and 30 cm⁻¹. In embodiments, the vaccine thin film has a surface area to volume ratio of between 5 and 20 cm⁻¹. In embodiments, the vaccine thin film has a surface area to volume ratio of between 5 and 15 cm⁻¹. In embodiments, the vaccine thin film has a surface area to volume ratio of between 5 and 10 cm⁻¹. In embodiments, the vaccine thin film has a surface area to volume ratio of about 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹.

In embodiments, the freezing rate of the droplets is between about 10 K/second and about 10⁵ K/second. In embodiments, the freezing rate of the droplets is between about 10 K/second and about 10⁴ K/second. In embodiments, the freezing rate of the droplets is between about 10 K/second and about 10³ K/second. In embodiments, the freezing rate of the droplets is between about 10² K/second and about 10³ K/second. In embodiments, the freezing rate of the droplets is between about 50 K/second and about 5×10² K/second. In embodiments, the freezing rate of the droplets is between 10 K/second and 10⁵ K/second. In embodiments, the freezing rate of the droplets is between 10 K/second and 10⁴ K/second. In embodiments, the freezing rate of the droplets is between 10 K/second and 10³ K/second. In embodiments, the freezing rate of the droplets is between 10² K/second and 10³ K/second. In embodiments, the freezing rate of the droplets is between 50 K/second and 5×10² K/second. In embodiments, the freezing rate of the droplets is about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, the freezing rate of the droplets is 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, each of the droplets freezes upon contact with the freezing surface in less than about 50, 75, 100, 125, 150, 175, 200, 250, 500, 1,000, or 2,000 milliseconds. In embodiments, each of the droplets freezes upon contact with the freezing surface in less than 50, 75, 100, 125, 150, 175, 200, 250, 500, 1,000, or 2,000 milliseconds.

In embodiments, the droplets have an average diameter between about 0.1 and about 5 mm, between about 4 and about 24° C. In embodiments, the droplets have an average diameter between about 2 and about 4 mm, between about 4 and about 24° C. In embodiments, the droplets have an average diameter between about 1 and about 4 mm, between about 4 and about 24° C. In embodiments, the droplets have an average diameter between about 2 and about 3 mm, between about 4 and about 24° C. In embodiments, the droplets have an average diameter between about 1 and about 3 mm, between about 4 and about 24° C. In embodiments, the droplets have an average diameter between about 1 and about 2 mm, between about 4 and about 24° C. In embodiments, the droplets have an average diameter between about 3 and about 4 mm, between about 4 and about 24° C. In embodiments, the droplets have an average diameter between 0.1 and 5 mm, between 4 and 24° C. In embodiments, the droplets have an average diameter between 2 and 4 mm, between 4 and 24° C. In embodiments, the droplets have an average diameter between 1 and 4 mm, between 4 and 24° C. In embodiments, the droplets have an average diameter between 2 and 3 mm, between 4 and 24° C. In embodiments, the droplets have an average diameter between 1 and 3 mm, between 4 and 24° C. In embodiments, the droplets have an average diameter between 1 and 2 mm, between 4 and 24° C. In embodiments, the droplets have an average diameter between 3 and 4 mm, between 4° C. and 24° C.

In embodiments, the method further includes removing the solvent (e.g., water or liquid) from the vaccine thin film to form a dry vaccine.

In embodiments, is a method of making a dry vaccine from a vaccine thin film (e.g., including a vaccine thin film made using a method as described herein), including removing the solvent (e.g., water or liquid) from the vaccine thin film to form a dry vaccine. In embodiments of the methods described herein, the dry vaccine is a dry vaccine as described herein, including in an aspect, embodiment, example, table, figure, or claim. In embodiments, a method of making a vaccine thin film or a method of making dry vaccine is used to make a dry vaccine as described herein, including in an aspect, embodiment, example, table, figure, or claim.

In embodiments, the removing of the solvent includes lyophilization. In embodiments, the removing of the solvent includes lyophilization at temperatures of 20° C. or less. In embodiments, the removing of the solvent includes lyophilization at temperatures of 25° C. or less. In embodiments, the removing of the solvent includes lyophilization at temperatures of 30° C. or less. In embodiments, the removing of the solvent includes lyophilization at temperatures of 35° C. or less. In embodiments, the solvent includes lyophilization at temperatures of 40° C. or less. In embodiments, the removing of the solvent includes lyophilization at temperatures of 25° C. or less. In embodiments, the removing of the solvent includes lyophilization at temperatures of about 20° C. or less. In embodiments, the removing of the solvent includes lyophilization at temperatures of about 15° C. or less. In embodiments, the solvent includes lyophilization at temperatures of about 10° C. or less. In embodiments, the removing of the solvent includes lyophilization at temperatures of about 5° C. or less.

In embodiments, the method further includes solvating the dry vaccine thereby forming a reconstituted liquid vaccine. A reconstituted liquid vaccine may also be called a solvated dry vaccine.

In embodiments, is a method of making a reconstituted liquid vaccine from a dry vaccine (e.g., including a dry vaccine made using a method as described herein), including solvating a dry vaccine and thereby forming a reconstituted liquid vaccine. In embodiments of the methods described herein, the dry vaccine is a dry vaccine as described herein, including in an aspect, embodiment, example, table, figure, or claim. In embodiments, a method of making a vaccine thin film, a method of making a dry vaccine, or a method of reconstituting a liquid vaccine is used to make a reconstituted liquid vaccine as described herein, including in an aspect, embodiment, example, table, figure, or claim.

In embodiments, the immunogenicity of the reconstituted liquid vaccine is at least 50% the immunogenicity of the liquid vaccine (prior to forming the dry vaccine from the liquid vaccine). In embodiments, the immunogenicity of the reconstituted liquid vaccine is at least 60% the immunogenicity of the liquid vaccine (prior to forming the dry vaccine from the liquid vaccine). In embodiments, the immunogenicity of the reconstituted liquid vaccine is at least 70% the immunogenicity of the liquid vaccine (prior to forming the dry vaccine from the liquid vaccine). In embodiments, the immunogenicity of the reconstituted liquid vaccine is at least 80% the immunogenicity of the liquid vaccine (prior to forming the dry vaccine from the liquid vaccine). In embodiments, the immunogenicity of the reconstituted liquid vaccine is at least 90% the immunogenicity of the liquid vaccine (prior to forming the dry vaccine from the liquid vaccine). In embodiments, the immunogenicity of the reconstituted liquid vaccine is at least 95% the immunogenicity of the liquid vaccine (prior to forming the dry vaccine from the liquid vaccine). In embodiments, the immunogenicity of the reconstituted liquid vaccine is at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% the immunogenicity of the liquid vaccine (prior to forming the dry vaccine from the liquid vaccine). In embodiments, the immunogenicity of the reconstituted liquid vaccine is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% the immunogenicity of the liquid vaccine (prior to forming the dry vaccine from the liquid vaccine).

In embodiments, the reconstituted liquid vaccine includes particles. In embodiments, the particles have an average diameter of between about 10 nm and about 2 μm. In embodiments, the particles have an average diameter of between about 20 nm and about 2 μm. In embodiments, the particles have an average diameter of between about 50 nm and about 2 μm. In embodiments, the particles have an average diameter of between about 100 nm and about 2 μm. In embodiments, the particles have an average diameter of between about 200 nm and about 2 μm. In embodiments, the particles have an average diameter of between about 500 nm and about 2 μm. In embodiments, the particles have an average diameter of between about 1 um and about 2 μm. In embodiments, the particles have an average diameter of between about 10 nm and about 1 μm. In embodiments, the particles have an average diameter of between about 10 nm and about 500 nm. In embodiments, the particles have an average diameter of between about 10 nm and about 250 nm. In embodiments, the particles have an average diameter of between about 10 nm and about 200 nm. In embodiments, the particles have an average diameter of between about 10 nm and about 100 nm. In embodiments, the particles have an average diameter of between about 10 nm and about 50 nm. In embodiments, the particles have an average diameter of between about 10 nm and about 20 nm. In embodiments, the particles have an average diameter of between about 20 nm and about 1 um. In embodiments, the particles have an average diameter of between about 50 nm and about 500 nm. In embodiments, the particles have an average diameter of between about 100 nm and about 250 nm. In embodiments, the particles have an average diameter of between about 100 nm and about 200 nm. In embodiments, the reconstituted liquid vaccine includes particles. In embodiments, the particles have an average diameter of between 10 nm and 2 μm. In embodiments, the particles have an average diameter of between 20 nm and 2 μm. In embodiments, the particles have an average diameter of between 50 nm and 2 μm. In embodiments, the particles have an average diameter of between 100 nm and 2 μm. In embodiments, the particles have an average diameter of between 250 nm and 2 μm. In embodiments, the particles have an average diameter of between 500 nm and 2 μm. In embodiments, the particles have an average diameter of between 1 um and 2 μm. In embodiments, the particles have an average diameter of between 10 nm and 1 μm. In embodiments, the particles have an average diameter of between 10 nm and 500 nm. In embodiments, the particles have an average diameter of between 10 nm and 200 nm. In embodiments, the particles have an average diameter of between 10 nm and 250 nm. In embodiments, the particles have an average diameter of between 10 nm and 100 nm. In embodiments, the particles have an average diameter of between 10 nm and 50 nm. In embodiments, the particles have an average diameter of between 10 nm and 20 nm. In embodiments, the particles have an average diameter of between 20 nm and 1 μm. In embodiments, the particles have an average diameter of between 50 nm and 500 nm. In embodiments, the particles have an average diameter of between 100 nm and 500 nm. In embodiments, the particles have an average diameter of between 100 nm and 250 nm.

In embodiments, the reconstituted liquid vaccine includes particles of similar average diameter as the liquid vaccine (prior to forming the dry vaccine from the liquid vaccine) particles. In embodiments, the reconstituted liquid vaccine includes particles having an average diameter within 5% of the average diameter of particles in the liquid vaccine (prior to forming the dry vaccine from the liquid vaccine). In embodiments, the reconstituted liquid vaccine includes particles having an average diameter within 10% of the average diameter of particles in the liquid vaccine (prior to forming the dry vaccine from the liquid vaccine). In embodiments, the reconstituted liquid vaccine includes particles having an average diameter within 20% of the average diameter of particles in the liquid vaccine (prior to forming the dry vaccine from the liquid vaccine). In embodiments, the reconstituted liquid vaccine includes particles having an average diameter within 30% of the average diameter of particles in the liquid vaccine (prior to forming the dry vaccine from the liquid vaccine). In embodiments, the reconstituted liquid vaccine includes particles having an average diameter within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 or 60% of the average diameter of particles in the liquid vaccine (prior to forming the dry vaccine from the liquid vaccine). In embodiments, the reconstituted liquid vaccine includes particles having an average diameter within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 95, 90, 95, 96, 97, 98, 99 or 100% of the average diameter of particles in the liquid vaccine (prior to forming the dry vaccine from the liquid vaccine).

In embodiments, the solvating of the dry vaccine is at least one day after preparing the dry vaccine from the liquid vaccine (e.g., the dry vaccine is stored for at least one day). In embodiments, the solvating of the dry vaccine is at least two days after preparing the dry vaccine from the liquid vaccine (e.g., the dry vaccine is stored for at least two days). In embodiments, the solvating of the dry vaccine is at least three days after preparing the dry vaccine from the liquid vaccine (e.g., the dry vaccine is stored for at least three days). In embodiments, the solvating of the dry vaccine is at least one week after preparing the dry vaccine from the liquid vaccine (e.g., the dry vaccine is stored for at least one week). In embodiments, the solvating of the dry vaccine is at least two weeks after preparing the dry vaccine from the liquid vaccine (e.g., the dry vaccine is stored for at least two weeks). In embodiments, the solvating of the dry vaccine is at least one month after preparing the dry vaccine from the liquid vaccine (e.g., the dry vaccine is stored for at least one month). In embodiments, the solvating of the dry vaccine is at least two months after preparing the dry vaccine from the liquid vaccine(e.g., the dry vaccine is stored for at least two months). In embodiments, the solvating of the dry vaccine is at least three months after preparing the dry vaccine from the liquid vaccine (e.g., the dry vaccine is stored for at least three months). In embodiments, the solvating of the dry vaccine is at least six months after preparing the dry vaccine from the liquid vaccine (e.g., the dry vaccine is stored for at least six months). In embodiments, the solvating of the dry vaccine is at least one year after preparing the dry vaccine from the liquid vaccine (e.g., the dry vaccine is stored for at least one year). In embodiments, the solvating of the dry vaccine is at least two years after preparing the dry vaccine from the liquid vaccine (e.g., the dry vaccine is stored for at least two years). In embodiments, the solvating of the dry vaccine is at least three years after preparing the dry vaccine from the liquid vaccine (e.g., the dry vaccine is stored for at least three years). In embodiments, the solvating of the dry vaccine is at least five years after preparing the dry vaccine from the liquid vaccine (e.g., the dry vaccine is stored for at least five years). In embodiments, the solvating of the dry vaccine is at least ten years after preparing the dry vaccine from the liquid vaccine (e.g., the dry vaccine is stored for at least ten years).

In embodiments, prior to the solvating of the dry vaccine, the dry vaccine is stored at about 4° C. for at least 99% of the time. In embodiments, prior to the solvating of the dry vaccine, the dry vaccine is stored at less than 4° C. for at least 99% of the time. In embodiments, prior to the solvating of the dry vaccine, the dry vaccine is stored at less than 0° C. for at least 99% of the time. In embodiments, prior to the solvating of the dry vaccine, the dry vaccine is stored at between 20 and 24° C. for at least 99% of the time. In embodiments, prior to the solvating of the dry vaccine, the dry vaccine is stored at between 4 and 24° C. for at least 99% of the time. In embodiments, prior to the solvating of the dry vaccine, the dry vaccine is stored at between 0 and 24° C. for at least 99% of the time. In embodiments, prior to the solvating of the dry vaccine, the dry vaccine is stored at between 4 and 40° C. for at least 99% of the time. In embodiments, prior to the solvating of the dry vaccine, the dry vaccine is stored at between 0 and 40° C. for at least 99% of the time. In embodiments, prior to the solvating of the dry vaccine, the dry vaccine is stored at about 4° C. for at least 90% of the time. In embodiments, prior to the solvating of the dry vaccine, the dry vaccine is stored at less than 4° C. for at least 90% of the time. In embodiments, prior to the solvating of the dry vaccine, the dry vaccine is stored at less than 0° C. for at least 90% of the time. In embodiments, prior to the solvating of the dry vaccine, the dry vaccine is stored at less than −20° C. for at least 90% of the time. In embodiments, prior to the solvating of the dry vaccine, the dry vaccine is stored at between 20 and 24° C. for at least 90% of the time. In embodiments, prior to the solvating of the dry vaccine, the dry vaccine is stored at between 4 and 24° C. for at least 90% of the time. In embodiments, prior to the solvating of the dry vaccine, the dry vaccine is stored at between 0 and 24° C. for at least 90% of the time. In embodiments, prior to the solvating of the dry vaccine, the dry vaccine is stored at between 4 and 40° C. for at least 90% of the time. In embodiments, prior to the solvating of the dry vaccine, the dry vaccine is stored at between 0 and 40° C. for at least 90% of the time.

In embodiments, upon solvating the dry vaccine the resulting reconstituted liquid vaccine remains homogeneous. As used in reference to the status of a reconstituted liquid vaccine, the term “homogenous” refers to a lack of a significant amount of aggregation and/or precipitation forming, such that the reconstituted liquid vaccine does not include solid matter that is not evenly dispersed (e.g., solid matter visible to the naked eye, solid matter that settles in the liquid, solid matter that was not apparent in a liquid vaccine prior to formation of the dry vaccine and reconstitution, precipitate that was not present in the liquid vaccine prior to formation of the dry vaccine). A homogenous reconstituted liquid sample may include particles/droplets of the O/W emulsion adjuvant (e.g., that are suspended or dispersed in the reconstituted liquid vaccine). In embodiments, upon solvating the dry vaccine the resulting reconstituted liquid vaccine remains homogeneous for at least one day. In embodiments, upon solvating the dry vaccine the resulting reconstituted liquid vaccine remains homogeneous for at least two days. In embodiments, upon solvating the dry vaccine the resulting reconstituted liquid vaccine remains homogeneous for at least three days. In embodiments, upon solvating the dry vaccine the resulting reconstituted liquid vaccine remains homogeneous for at least one week. In embodiments, upon solvating the dry vaccine the resulting reconstituted liquid vaccine remains homogeneous for at least two weeks. In embodiments, upon solvating the dry vaccine the resulting reconstituted liquid vaccine remains homogeneous for at least one month. In embodiments, upon solvating the dry vaccine the resulting reconstituted liquid vaccine remains homogeneous for at least three months. In embodiments, upon solvating the dry vaccine the resulting reconstituted liquid vaccine remains homogeneous for at least six months. In embodiments, upon solvating the dry vaccine the resulting reconstituted liquid vaccine remains homogeneous for at least one year. In embodiments, upon solvating the dry vaccine the resulting reconstituted liquid vaccine does not form a precipitate (e.g., solid matter visible to the naked eye, solid matter that settles in the liquid, solid matter that was not apparent in a liquid vaccine prior to formation of the dry vaccine and reconstitution, precipitate that was not present in the liquid vaccine prior to formation of the dry vaccine). In embodiments, upon solvating the dry vaccine the resulting reconstituted liquid vaccine does not form a precipitate for at least one day. In embodiments, upon solvating the dry vaccine the resulting reconstituted liquid vaccine does not form a precipitate for at least two days. In embodiments, upon solvating the dry vaccine the resulting reconstituted liquid vaccine does not form a precipitate for at least three days. In embodiments, upon solvating the dry vaccine the resulting reconstituted liquid vaccine does not form a precipitate for at least one week. In embodiments, upon solvating the dry vaccine the resulting reconstituted liquid vaccine does not form a precipitate for at least two weeks. In embodiments, upon solvating the dry vaccine the resulting reconstituted liquid vaccine does not form a precipitate for at least one month. In embodiments, upon solvating the dry vaccine the resulting reconstituted liquid vaccine does not form a precipitate for at least three months. In embodiments, upon solvating the dry vaccine the resulting reconstituted liquid vaccine does not form a precipitate for at least six months. In embodiments, upon solvating the dry vaccine the resulting reconstituted liquid vaccine does not form a precipitate for at least one year. In embodiments, the precipitate includes particles having an average diameter greater than 50 μm. In embodiments, the precipitate includes particles having an average diameter greater than 100 μm. In embodiments, the precipitate includes particles having an average diameter greater than 200 μm. In embodiments, the precipitate includes particles having an average diameter greater than 300 μm. In embodiments, the precipitate includes particles having an average diameter greater than 400 μm. In embodiments, the precipitate includes particles having an average diameter greater than 500 μm. In embodiments, the precipitate includes particles having an average diameter greater than 600 μm. In embodiments, the precipitate includes particles having an average diameter greater than 700 μm. In embodiments, the precipitate includes particles having an average diameter greater than 800 μm. In embodiments, the precipitate includes particles having an average diameter greater than 900 μm. In embodiments, the precipitate includes particles having an average diameter greater than 1000 μm. In embodiments, the precipitate includes particles having an average diameter greater than about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate includes particles having an average diameter of about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate includes particles having an average diameter greater than 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate (that is not formed) includes particles having an average diameter of 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm. In embodiments, the precipitate (that is not formed) includes at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the total antigenic protein and the O/W emulsion adjuvant in the reconstituted liquid vaccine.

In embodiments, the liquid vaccine includes a commercially available vaccine. In embodiments, the liquid vaccine is a commercially available vaccine. In embodiments, the liquid vaccine has received market approval from the US FDA or the corresponding authority in another country. In embodiments, the liquid vaccine is a vaccine for the treatment of diphtheria, tetanus, pertussis, influenza, pneumonia, otitis media, bacteremia, meningitis, hepatitis, cirrhosis, anthrax poisoning, botulism, rabies, warts, poliomyelitis, Japanese encephalitis, Coronavirus diseases, or cancer. In embodiments, the liquid vaccine is a vaccine for the treatment of infection by Clostridium tetani, Clostridium botulinum, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, coronavirus, or Poliovirus. In embodiments, the liquid vaccine is a vaccine for the prevention or treatment of a type of tumor. In embodiments, the liquid vaccine includes a commercially available vaccine and another component not included in the commercially available vaccine (e.g., an excipient (e.g., trehalose)).

In an aspect is provided a method of treating a disease in a subject in need of such treatment, the method including administering a therapeutically effective amount of a solvated dry vaccine as described herein (e.g., in an aspect, embodiment, example, table, figure, or claims) (e.g., a reconstituted liquid vaccine as described herein) to the patient.

In embodiments, the disease is diphtheria, tetanus, pertussis, influenza, pneumonia, otitis media, bacteremia, meningitis, hepatitis, cirrhosis, anthrax poisoning, rabies, warts, poliomyelitis, Japanese encephalitis, coronavirus diseases, or cancer. In embodiments, the disease is caused by an infectious agent. In embodiments, the infectious agent is a bacterium. In embodiments, the infectious agent is a virus. In embodiments, the infectious agent is Clostridium tetani, Clostridium botulinum, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, coronavirus, or Poliovirus. In some embodiments, the disease is a type of tumor.

In an aspect is provided a method of treating a disease in a patient in need of such treatment, the method including administering a therapeutically effective amount of dry vaccine as described herein (e.g., in an aspect, embodiment, example, table, figure, or claims) (e.g., a reconstituted liquid vaccine as described herein) to the patient.

In embodiments, the disease is diphtheria, tetanus, pertussis, influenza, pneumonia, otitis media, bacteremia, meningitis, hepatitis, cirrhosis, anthrax poisoning, botulism, rabies, warts, poliomyelitis, Japanese encephalitis, coronavirus diseases, or cancer. In embodiments, the disease is caused by an infectious agent. In embodiments, the infectious agent is a bacterium. In embodiments, the infectious agent is a virus. In embodiments, the infectious agent is Clostridium tetani, Clostridium botulinum, Streptococcus pneumonia, Hepatitis A, Hepatitis B, Haemophilus influenza, Corynebacterium diphtheria, Bordetella pertussis, Human papillomavirus, Bacillus anthracis, Rabies virus, Japanese encephalitis virus, coronavirus, or Poliovirus.

In embodiments, the dry vaccine is administered by inhalation, intradermally, orally, or vaginally. In embodiments, the dry vaccine is administered through the nasal mucosa, bronchoalveolar mucosa, or gastrointestinal mucosa.

In embodiments, the method is a method described herein, including in an aspect, embodiment, example, table, figure, or claim. Provided herein is a method of preparing a dry vaccine including a method of preparing a vaccine thin film as described herein (including in an aspect, embodiment, example, table, figure, or claim) and a method of removing a solvent from a vaccine thin film as described herein (including in an aspect, embodiment, example, table, figure, or claim). Provided herein is a method of preparing a reconstituted dry vaccine including a method of preparing a dry vaccine as described herein (including in an aspect, embodiment, example, table, figure, or claim), a method of preparing a vaccine thin film as described herein (including in an aspect, embodiment, example, table, figure, or claim) and a method of removing a solvent from a vaccine thin film as described herein (including in an aspect, embodiment, example, table, figure, or claim).

In embodiments, to form a powder vaccine, an aqueous vaccine composition is first frozen to form a frozen vaccine composition, then the frozen water is removed to form the frozen vaccine. A fast-freezing process is used to form the frozen vaccine composition. A fast-freezing process, as used herein, is a process that can freeze a thin film of liquid (less than about 5000 microns) in a time of less than or equal to about 5 seconds. Examples of fast freezing processes that may be used include thin film freezing (TFF), spray freeze-drying (SFD), or spray freezing into liquids (SFL). In the TFF process liquid droplets fall from a given height and impact, spread, and freeze on a cooled solid substrate. Typically, the substrate is a metal drum that is cooled to below 250 K, or below 200 K or below 150 K. On impact the droplets that are deformed into thin films freeze in a time of between about 70 ms and 5000 ms. The frozen thin films may be removed from the freezing surface by a stainless-steel blade mounted along the rotating drum surface. The frozen thin films are collected in liquid nitrogen to maintain in the frozen state. Further details regarding thin film freezing processes may be found in the paper to Engstrom et al. (Engstrom et al., 2008), which is incorporated herein by reference.

Water (e.g., frozen water) is removed from the frozen vaccine composition to produce a vaccine powder. Water (e.g., frozen water) may be removed by a lyophilization process or a freeze-drying process. Water may also be removed by an atmospheric freeze-drying process.

The resulting vaccine powder can be readily reconstituted to form a stable dispersion without significant loss of stability or activity. The vaccine powder may be transported and stored in a wide range of temperatures without concern of accidental exposure to freezing conditions. In addition, the vaccine powder may also be stored at room temperature, which will potentially decrease the costs of vaccines. In fact, it is generally less costly to transport dry solid powder than liquid.

Currently human vaccines (e.g., marketed and/or approved human vaccines, such as FDA approved human vaccines) that have adjuvant are all administered by needle-syringe-based injections. It would be beneficial to patients and the healthcare system if the vaccines were administered non-invasively without hypodermic needles. The dried vaccine powder can potentially be administered by an alternative route such as, but not limited to, inhalation as a dried powder, intranasally as a dried powder, intradermally using a solid jet injection device (e.g., powder jet injector), orally in tablets or capsules, buccally in buccal tablets or films, or vaginally using a special vaginal drug delivery device. The above-mentioned routes of administration are not only more convenient and friendly to patients, but more importantly they can enable the induction of mucosal immune responses. Functional antibodies in the mucosal secretion (e.g., nasal mucus, bronchoalveolar mucus, or the gastrointestinal mucus) of a host can effectively neutralize pathogens or toxins even before they enter the host.

Described herein are compositions and methods for preparing a vaccine thin film or a dry vaccine by spraying or dripping droplets of a liquid vaccine such that the vaccine is exposed to an vapor-liquid interface of less than 500 cm⁻¹ area/volume (e.g., less than 50, 100, 150, 200, 250, 300, 400) and contacting the droplet with a freezing surface having a temperature lower than the freezing temperature of the liquid vaccine (e.g., has a temperature differential of at least 30° C. between the droplet and the surface), wherein the surface freezes the droplet into a thin film with a thickness of less than 500 micrometers (e.g., less than 450, 400, 350, 300, 250, 200, 150, 100, or 50 micrometers) and a surface area to volume between 5 to 500 cm⁻¹. In embodiments, the method may further include the step of removing the liquid (e.g., solvent, water) from the frozen material to form a dry vaccine (e.g., particles). In embodiments, the droplets freeze upon contact with the surface in less than 50, 75, 100, 125, 150, 175, 200, 250, 500, 1,000 or 2,000 milliseconds. In embodiments, the droplets freeze upon contact with the surface in less than 50 or 150 milliseconds. In embodiments, the droplet has a diameter between 2 and 5 mm at room temperature. In embodiments, the droplet forms a thin film on the freezing surface of between 50 and 5000 micrometers in thickness. In embodiments, the droplets have a cooling rate of between 50-250 K/s. In embodiments, the particles of the dry vaccine, after liquid (e.g., solvent or water) removal, have a surface area of at least 10, 15, 25, 50, 75, 100, 125, 150 or 200 m²/gr (e.g., surface area of 10, 15, 25, 50, 75, 100, 125, 150 or 200 m²/gr). Minimizing gas-liquid interface can improve protein stability by limiting the amount of protein that can adsorb to the interface.

In embodiments, the droplets may be delivered to the cold or freezing surface in a variety of manners and configurations. In embodiments, the droplets may be delivered in parallel, in series, at the center, middle or periphery or a platen, platter, plate, roller, conveyor surface. In embodiments, the freezing or cold surface may be a roller, a belt, a solid surface, circular, cylindrical, conical, oval and the like that permit for the droplet to freeze. For a continuous process a belt, platen, plate or roller may be particularly useful. In embodiments, the frozen droplets may form beads, strings, films or lines of frozen liquid vaccine. In embodiments, the effective ingredient is removed from the surface with a scraper, wire, ultrasound or other mechanical separator prior to the lyophilization process. Once the material is removed from the surface of the belt, platen, roller or plate the surface is free to receive additional material.

In embodiments, the surface is cooled by a cryogenic solid, a cryogenic gas, a cryogenic liquid or a heat transfer fluid capable of reaching cryogenic temperatures or temperatures below the freezing point of the liquid vaccine (e.g., at least 30° C. less than the temperature of the droplet). In embodiments, the liquid vaccine further includes one or more excipients selected from sugars, phospholipids, surfactants, polymeric surfactants, vesicles, polymers, including copolymers and homopolymers and biopolymers, dispersion aids, and serum albumin In embodiments, the temperature differential between the droplet and the surface is at least 50° C. In embodiments, the excipients or stabilizers that can be included in the liquid vaccines that are to be frozen as described herein include: cryoprotectants, lyoprotectants, surfactants, fillers, stabilizers, polymers, protease inhibitors, antioxidants and absorption enhancers. Specific nonlimiting examples of excipients that may be included in the vaccines described herein include: Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol.

In embodiments, the method may further include the step of removing the liquid (e.g., solvent or water) from the frozen liquid vaccine to form a dry vaccine. In embodiments, the solvent further includes at least one or more excipient or stabilizers selected from, e.g., phospholipids, surfactants, polymeric surfactants, vesicles, polymers, including copolymers and homopolymers and biopolymers, dispersion aids, and serum albumin In embodiments, the temperature differential between the solvent and the surface is at least 50° C.

In embodiments, the resulting powder can be redispersed into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals (e.g., humans).

In embodiments, is described a single-step, single-vial method for preparing a vaccine thin film or dry vaccine by reducing the temperature of a vial wherein the vial has a temperature below the freezing temperature of a liquid vaccine (e.g., a temperature differential of at least 30° C. between the liquid vaccine and the vial) and spraying or dripping droplets of a liquid vaccine directly into the vial such that the antigenic protein of the liquid vaccine is exposed to a vapor-liquid interface of less than 500 cm⁻¹ area/volume, wherein the surface freezes the droplet into a thin film with a thickness of less than 5000 micrometers and a surface area to volume between 5 to 500 cm⁻¹. In embodiments, the droplets freeze upon contact with the surface in less than about 50, 75, 100, 125, 150, 175, 200, 250, 500, 1,000 or 3,000 milliseconds (e.g., in about 50, 75, 100, 125, 150, 175, 200, 250, 500, 1,000 or 3,000 milliseconds), and may freeze upon contact with the surface in about 50 or 150 to 500 milliseconds. In embodiments, a droplet has a diameter between 0.1 and 5 mm at room temperature (e.g., a diameter between 2 and 4 mm at room temperature). In embodiments, the droplet forms a thin film on the surface of between 50 and 500 micrometers in thickness. In embodiments, the droplets have a cooling rate of between 50-250 K/s. In embodiments, the vial may be cooled by a cryogenic solid, a cryogenic gas, a cryogenic liquid, a freezing fluid, a freezing gas, a freezing solid, a heat exchanger, or a heat transfer fluid capable of reaching cryogenic temperatures or temperatures below the freezing point of the liquid vaccine. In embodiments, the vial may be rotated as the spraying or droplets are delivered to permit the layering or one or more layers of the liquid vaccine. In embodiments, the vial and the liquid vaccine are pre-sterilized prior to spraying or dripping. In embodiments, the step of spraying or dripping may be repeated to overlay one or more thin films on top of each other to fill the vial to any desired level up to totally full.

### IV. EXAMPLES

The following examples as well as the figures are included to demonstrate preferred embodiments of the disclosure. It should be appreciated by those of skill in the art that the techniques disclosed in the examples or figures represent techniques discovered by the inventors to function well in the practice of the disclosure and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1

### Dry Powder Compositions of AddaVax™ and AddaVax™-Adjuvanted Vaccines

Vaccine adjuvants are employed to enhance vaccines' immunogenicity and thus spare the antigens' dose, which could be crucial in the event of a pandemic. Vaccine adjuvants should be adequately formulated for maximum safety and efficacy. Adjuvant formulations include liposomes, virosomes, ISCOMs (i.e., immunostimulatory complex in which protein antigen is incorporated into saponin) and oil-in-water (O/W) emulsions. Emulsion-based adjuvants have enabled broadening of immune responses as well as dose sparing (Alving et al., 2012). Examples of O/W vaccine adjuvants include AS03, MF59® and AddaVax™. MF59® is an O/W emulsion containing squalene oil (4.3%) in citrate buffer stabilized with non-ionic surfactants (i.e., Tween 80 (0.5%) and Span 85 (0.5%)), having a mean droplet size of 160 nm (Ko & Kang, 2012). AddaVax™ is also a squalene-based O/W nanoemulsion with a similar formulation and mean droplet size to that of MF59®. Both MF59® and AddaVax™ can enhance cellular and humoral immune responses (van Diepen et al., 2018). MF59® is licensed for use in pandemic and seasonal influenza vaccines (e.g., Fluad®, Celtura® and Focetria®) in many countries (Ko & Kang, 2012). These vaccines should be stored at 2° C.-8° C. and should not be frozen.

Herein, the inventors report that dry powder compositions can conserve the particle size distribution, such as by limiting particle size change within 60% of the starting material and antigen integrity of O/W nanoemulsion-adjuvanted vaccines. The dry powder compositions are expected to address vaccine sensitivity to accidental freezing and/or heat. AddaVax™ (InvivoGen, San Diego, Calif.) was employed as an adjuvant and ovalbumin (OVA, Sigma Aldrich, St Louis, Mo.) and lysozyme as model antigens at different doses (i.e., 6, 12 and 50 μg). Various excipients (i.e., trehalose, sucrose and mannitol) were used at different concentrations (i.e., 50, 100, 125, 250 and 500 mg/mL). Liquid OVA/AddaVax™ formulations (Table 1) were converted into dry powders using thin-film freeze-drying (TFFD) technology. The formulations were dropped onto a cryogenically cooled surface having a temperature of −20° C., −35° C., −50° C., −100° C. or −180° C. to form frozen thin films. The frozen films were then lyophilized using a VirTis Advantage bench top tray lyophilizer (The VirTis Company, Inc. Gardiner, N.Y.). Lyophilization was performed over about 50 h at pressures ≤100 mTorr. The shelf temperature was maintained at −35° C. for 30 h then gradually ramped to +20° C. over about 16 h. Throughout the secondary drying phase, the vials were kept at +20° C. for additional 4 h. The vials were then stored at room temperature for future use.

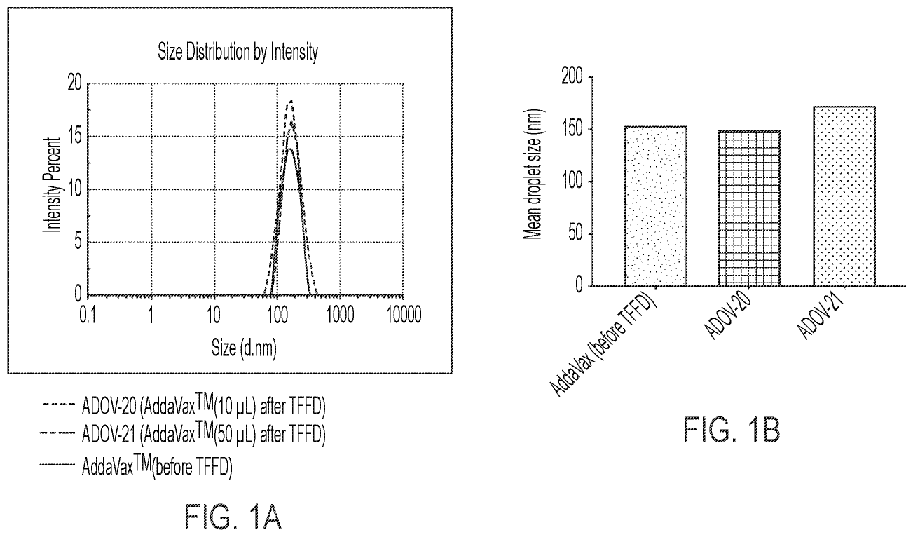

As shown in the FIGS. 1A-B, the mean droplet size and size distribution of the nanoemulsion-based adjuvant (i.e., AddaVax™) was maintained after it was subjected to TFFD and reconstitution, especially for formulation ADOV-20. This observation prompted the inventors to test the effect of antigen incorporation as well as other formulation and processing parameters on the droplet size distribution of the TFFD-processed powder of vaccines adjuvanted with AddaVax™.

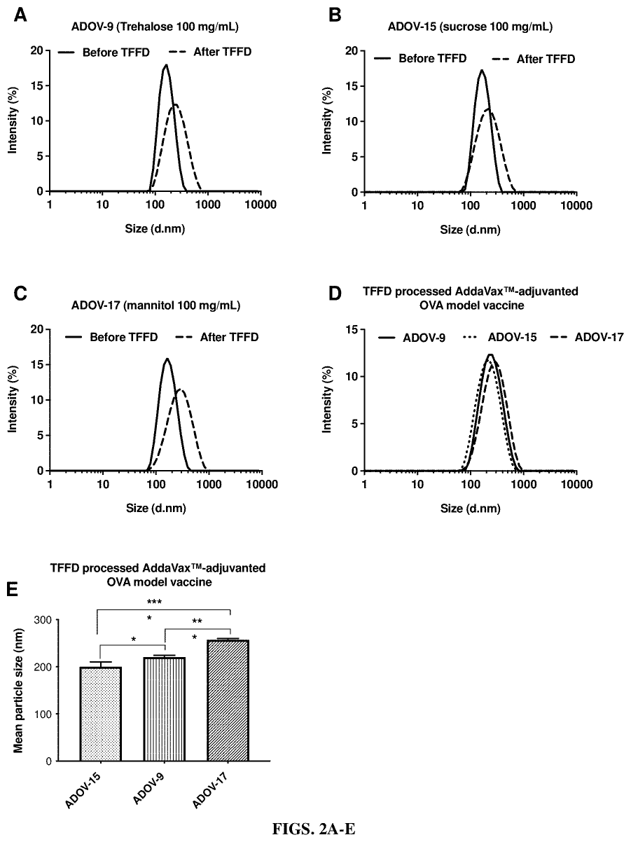

As shown in FIGS. 2A-E, all screened sugars/sugar alcohols almost maintained the mean particle size of AddaVax™-adjuvanted OVA vaccine. However, sucrose (ADOV-15) was more effective in maintaining the particle size distribution after TFFD compared to trehalose (p<0.05) or mannitol (p<0.0001). Mannitol was the least effective.

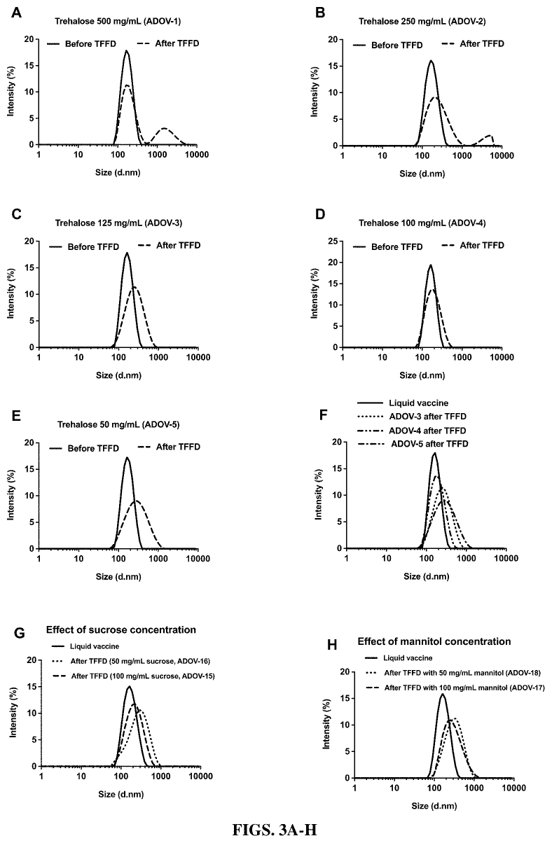

As shown in FIGS. 3A-B, high concentration levels of trehalose (i.e., 500 and 250 mg/mL) failed to maintain an acceptable particle size distribution of AddaVax™-adjuvanted OVA vaccine. On the other hand, relatively lower concentration levels of trehalose (i.e., 125, 100 and 50 mg/mL) was better in maintaining the particle size distribution of the vaccine better. As shown in FIGS. 3F-H, the excipients better maintained the particle size distribution at a concentration level of 100 mg/mL compared to 125 mg/mL; however, at a concentration level of 50 mg/mL the excipient was less efficient in maintaining the particle size distribution compared to a concentration level of 100 mg/mL. As shown in FIGS. 4A-C, conventional shelf freeze-drying had a deleterious effect on the particle size distribution of the AddaVax™-adjuvanted model vaccine and led to the generation of an extra group of particulates in the range of 1000 nm (FIG. 4A). On the other hand, TFFD kept the vaccine's particle size distribution within acceptable range. Thus, unlike conventional shelf freeze-drying, TFFD technology can be used to convert AddaVax™-adjuvanted liquid vaccines into dry powders while having a minimum effect on their particle size distribution. SDS-PAGE showed neither aggregation nor fragmentation of the OVA protein after the AddaVax™-adjuvanted OVA was subjected to TFFD and reconstitution (FIG. 4C).

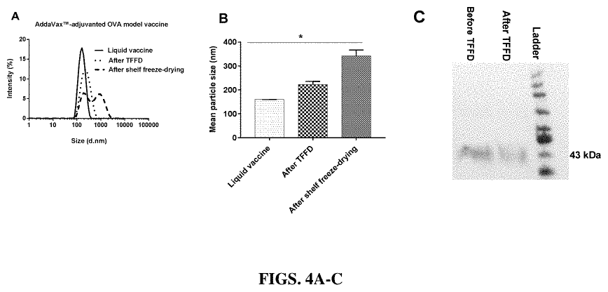

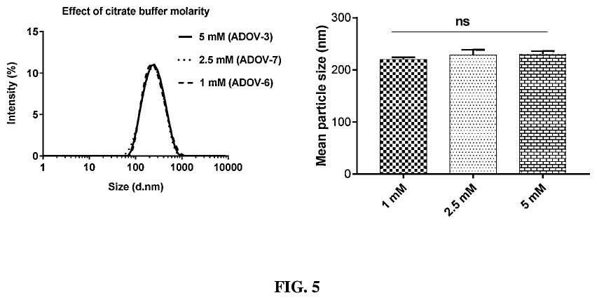

As shown in FIG. 5, the buffer molarity did not have any significant effect on the particle size distribution of the model vaccine. Thus, a relatively wide range of buffer molarities can be used to successfully process the AddaVax™-adjuvanted vaccines into dry powders using TFFD technology.

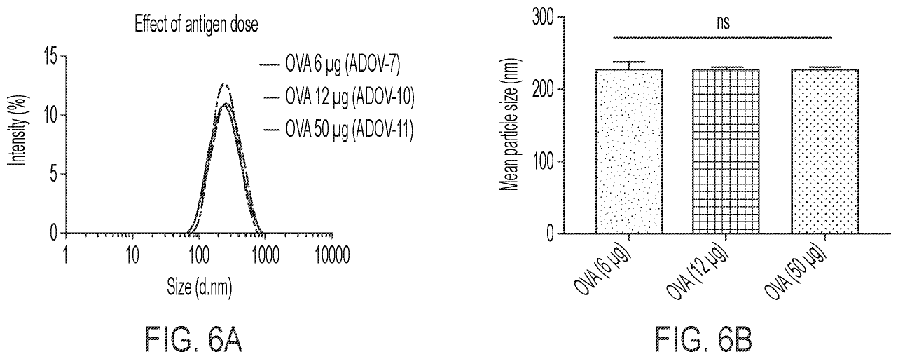

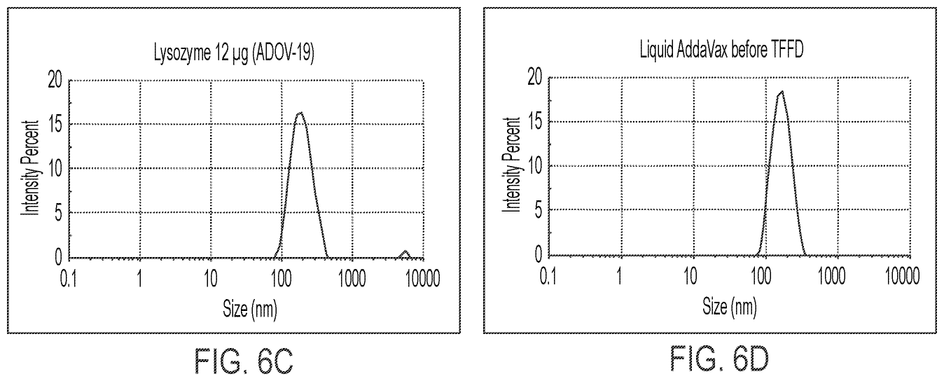

As shown in FIGS. 6A-B, the antigen level in the formulation had no significant effect on the particle size distribution of the model vaccine. In FIGS. 6C-D, the particle size distribution of AddaVax™-adjuvanted vaccine was even better maintained when lysozyme was employed as a model antigen at a dose of 12 μg. Overall, it appeared that the droplet size distribution was better maintained when lysozyme was employed as a model antigen as compared to OVA. Thus, the type of antigen can affect the particle size distribution of TFFD-processed vaccine powders after reconstitution, likely related to the isoelectric point (pI) of the antigens. For example, the pI of OVA is 5.19, 11.1 for lysozyme. The zeta potential of the AddaVax™ in water was −10.1 mV, −2.69 mV in citrate buffer.

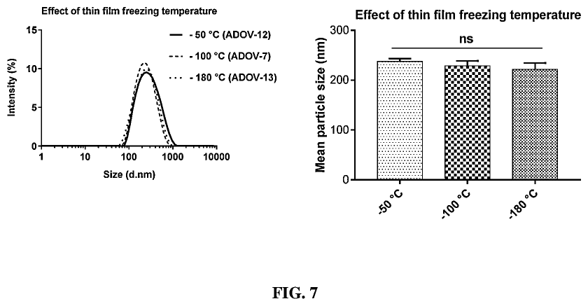

As shown in FIG. 7, the processing temperature had no effect on the particle size distribution of the model vaccine. Thus, AddaVax™-adjuvanted liquid vaccines can be successfully processed into dry powders using TFFD at a wide range of processing temperatures. FIG. 8 shows that the vaccine powder had a glass transition temperature of over 100° C., indicating that it could potentially be stored at room temperature. The moisture content in the dry vaccine in FIG. 8 was determined to be 4.9±0.3%. Prolonging the secondary drying during the sublimation step is expected to reduce the moisture content to lower levels, if needed.

### Example 2

### Dry Powder Compositions of Fluad Quadrivalent Vaccine

Fluad Quadrivalent® (Seqirus Inc., Holly Springs, N.C.) is an MF59-adjuvanted vaccine for active immunization against influenza disease caused by influenza viruses subtypes A and types B whose antigens are contained in the vaccine. Fluad Quadrivalent® vaccine contains MF59 and the hemagglutinin (HA) proteins of four influenza strains at 15 μg per 0.5 mL each (FDA 2022).

Materials and Methods. Trehalose in citrate buffer (2.5 mM, pH 6.5) (50 μL) at 125 mg/mL was employed as a stabilizer. The sugar was mixed with 50 μL of the Fluad Quadrivalent vaccine and the resultant liquid vaccine formulation was then frozen to thin-films at −100° C. and dried as described in Example-1. Thin-film freeze-dried vaccine powder was then reconstituted in water, and particle size distribution, PDI and zeta potential values were measured using a Malvern Zeta Sizer Nano ZS after dilution with water. SDS-PAGE analysis was used to explore the integrity of HA proteins after TFFD and reconstitution. Briefly, 10 μL of the reconstituted Fluad Quadrivalent vaccine was mixed with Laemmli Sample Buffer (Bio-Rad, Hercules, Calif.) and β-mercaptoethanol (2%, v/v, Sigma-Aldrich). The Sample was heated at 95° C. for 5 mM prior to loading onto 4-20% Mini-PROTEAN® TGX™ precast polyacrylamide gel (Bio-Rad). Gel electrophoresis was done at 100 V for 90 mM. Gel was stained in a Bio-Safe™ Coomassie G-250

Stain (Bio-Rad).

The integrity and functionality of the HA proteins in the vaccine dry powder prepared using TFFD were evaluated using a standard hemagglutination assay using chicken erythrocytes as previously described (Mandon et al., 2020). Briefly, 50 μL of the reconstituted vaccine dry powder was two-fold serially diluted in phosphate buffered saline (PBS, 10 mM, pH 7.2) in U-bottom 96-well plates. Then, 50 μL of 1% chicken erythrocyte suspension (Rockland Immunochemicals, Inc., Limerick, Pa.) in PBS was added and samples were incubated at room temperature for 30 min Hemagglutination titers were reported as the reciprocal of the last dilution where hemagglutination was observed (i.e., absence of chicken erythrocyte precipitation) and were expressed in hemagglutination units (HAUs)/50 μL.

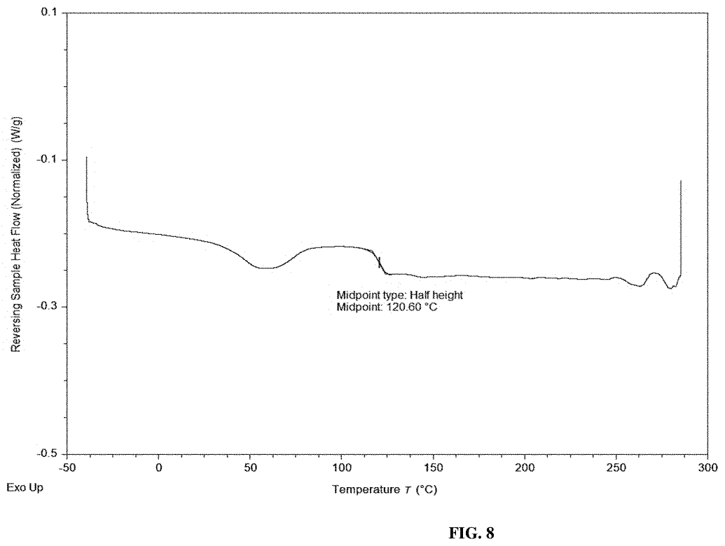

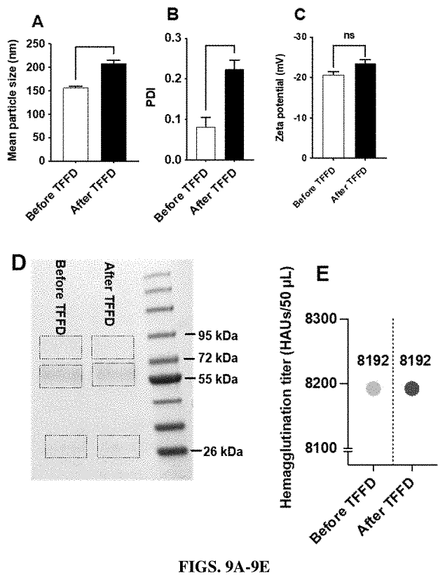

Results. As depicted in FIG. 9A-C, mean particle size and PDI of the reconstituted vaccine increased slightly, but the zeta potential of the vaccine particles was maintained. Importantly, the integrity of the antigens as indicated by the SDS-PAGE analysis (FIG. 9D) and the hemagglutination activity of the HA antigens in the vaccine (FIG. 9E) remained unchanged. These results further confirm the applicability of TFFD to converting vaccines adjuvanted with MF59 into dry powders.

### Example 3

### Dry Powder Compositions of AddaVax-Adjuvanted OVA Model Vaccine for Intranasal Delivery

Mucosal immunization (e.g., intranasal, pulmonary, and oral immunization) offers several advantages over other routes of vaccine delivery (AboulFotouh et al., 2022). For instance, it has high patient compliance, does not require specialized personnel for administration and therefore it increases the rate and reduces the cost of mass immunization, and eliminates needle-associated risk of injury and infection (Skwarczynski & Toth, 2020). Importantly, the mucosal routes (e.g., the respiratory tract) are the port of entry for many pathogens (e.g., influenza viruses and SARS-CoV-2 virus) (AboulFotouh et al., 2021). Thus, it could be expected that most of vaccines would be administered via mucosal routes; however, this is not the case. Mucosal vaccine delivery is challenged by the lack of approved mucosal vaccine adjuvants. Furthermore, the systemically-administered vaccine formulations and delivery systems are not compatible with the mucosal routes resulting in suboptimal mucosal and systemic immune responses (Skwarczynski & Toth, 2020). Therefore, new vaccine adjuvants and delivery systems have emerged to overcome this obstacle.

For example, the O/W nanoemulsion vaccine adjuvant MF59@ has been shown to be safe when administered intranasally in humans (Boyce et al., 2000). Intranasal immunization may be more effective than intramuscular administration for the prevention of influenza infection (Ren et al., 2017). Intranasal immunization is more advantageous than other mucosal routes in terms of rapid onset of action and direct access to the central nervous system (Salade et al., 2019). In this study we investigated the potential of AddaVax-adjuvanted OVA vaccine dry powder compositions prepared using TFFD for intranasal delivery. Dry powder formulations are more stable and can protect the adjuvant and the antigen against shearing-induced stress during spraying or nebulization of liquid formulations (Zhang et al., 2020).

Materials and Methods. Liquid formulation of AddaVax-adjuvanted OVA model vaccine (AddaVax/OVA) containing AddaVax (50 μL, InvivoGen, San Diego, Calif.), OVA (6 μg, Sigma-Aldrich) and D-trehalose dihydrate (Sigma-Aldrich) at a concentration of 125 mg/mL was prepared by simple mixing. The liquid vaccine formulation (100 μL) was subjected to TFFD as described in Example 1. The formulation was in citrate buffer (2.5 mM, pH 6.5) and frozen into thin-films at −100° C. Then, the powder particle size analysis was performed using Malvern Spraytec laser diffraction system (Malvern Ltd., Malvern, UK)). DeVilbiss Healthcare powder blower (DeVilbiss Healthcare LLC., Somerset, Pa.) was employed as the nasal spray devise. The complete time history profiles of the particle size distribution statistics (i.e., Dᵥ(10), Dᵥ(50), and Dᵥ(90)), percentage transmission (% T) and span at 6 cm from the tip of the powder blower were reported. The span is calculated as [(Dᵥ(90)-Dᵥ(10))/Dᵥ(50)] and reflects the width of size distribution. Dᵥ(10) is the volume diameter where 10% of the particles with diameters below this value reside, Dᵥ(50) is the volume diameter where 50% of the particles with diameters below or above this value reside, and Dᵥ(90) is the volume diameter where 90% of the particles with diameters below this value reside. Data were reported during the formation phase, the fully-developed phase, and the dissipation phase.

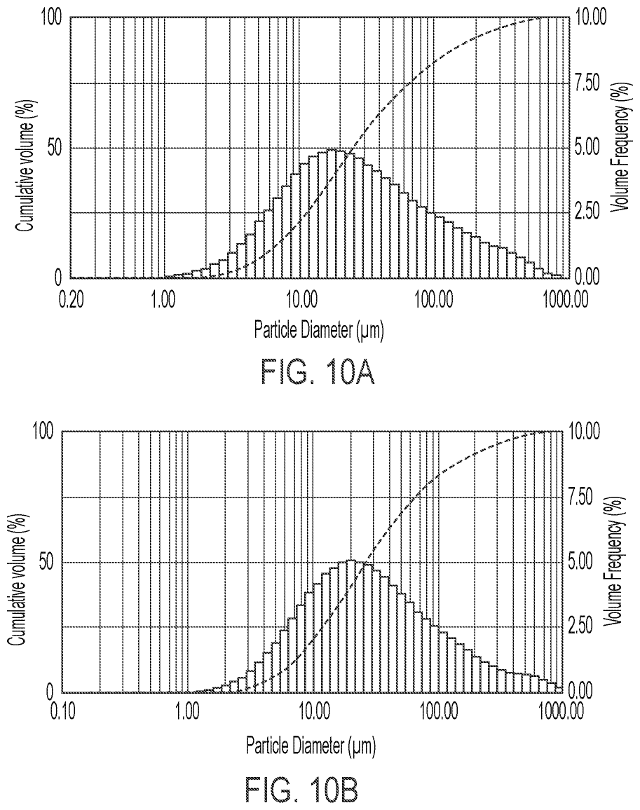

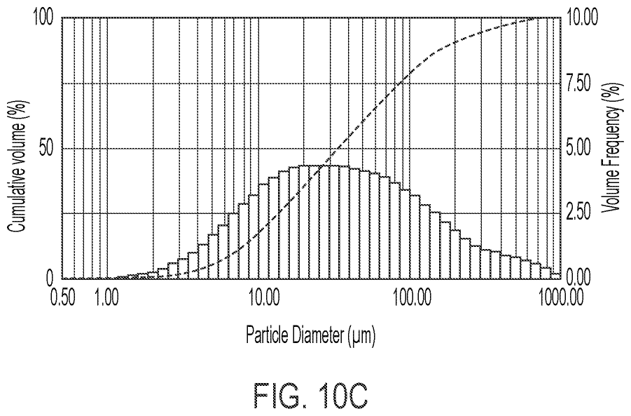

Results. The main particle size cut-off considered for intranasal delivery is 10 μm. Particles with a median diameter <10 μm can travel to the lower respiratory tract while particles (Salade et al., 2019). The median particle size of AddaVax/OVA dry powder prepared using TFFD ranges from 24 μm to 33 μm depending on the phase (Table 2 and FIG. 10). Therefore, when administered using a proper nasal dry powder sprayer, the powder can potentially deposit in the nasal cavity for eliciting the desired immune responses (Xu et al., 2020).

TABLE 1
Formulation compositions and processing temperatures.

| | AddaVax ™ | | | | | | | | | | |
|---|---|---|---|---|---|---|---|---|---|---|---|
| | | | | Sodium | Antigen | | | | | | Drum |
| | Sorbitan | Tween | Squalene | citrate | | Lyso- | | Excipient | | Total | temperature |
| | trioleate | 80 | oil | buffer | OVA | zyme | Excipient | mass | | volume | during TFF |
| Formulation | (mg) | (mg) | (mg) | (mg) | (μg) | (μg) | (concentration) | fraction | Buffer | (μL) | (° C.) |
| ADOV-1 | 0.25 | 0.25 | 2.5 | 0.1406 | 6 | — | Trehalose (500 mg/mL) | 0.886 | Citrate buffer | 100 | −100 |
| ADOV-2 | | | | | | | Trehalose (250 mg/mL) | 0.796 | (5 mM, pH 6.5) | | |
| ADOV-3 | | | | | | | Trehalose (125 mg/mL) | 0.661 | | | |
| ADOV-4 | | | | | | | Trehalose (100 mg/mL) | 0.609 | | | |
| ADOV-5 | | | | | | | Trehalose (50 mg/mL) | 0.438 | | | |
| ADOV-6 | | | | | | | Trehalose (125 mg/mL) | 0.762 | Citrate buffer | | |
| | | | | | | | | | (1 mM, pH 6.5) | | |
| ADOV-7 | | | | | | | Trehalose (125 mg/mL) | 0.721 | Citrate buffer | | |
| | | | | | | | | | (2.5 mM, pH 6.5) | | |
| ADOV-8 | | | | | | | Trehalose (100 mg/mL) | 0.719 | Citrate buffer | | |
| | | | | | | | | | (1 mM, pH 6.5) | | |
| ADOV-9 | | | | | | | Trehalose (100 mg/mL) | 0.673 | Citrate buffer | | |
| | | | | | | | | | (2.5 mM, pH 6.5) | | |
| ADOV-10 | | | | | 12 | | Trehalose (125 mg/mL) | 0.714 | Citrate buffer | | |
| ADOV-11 | | | | | 50 | | Trehalose (125 mg/mL) | 0.663 | (2.5 mM, pH 6.5) | | |
| ADOV-12 | | | | | 6 | | Trehalose (125 mg/mL) | 0.721 | | | −50 |
| ADOV-13 | | | | | | | Trehalose (125 mg/mL) | 0.721 | | | −180 |
| ADOV-14* | 1.25 | 1.25 | 12.5 | 0.703 | 12 | | Trehalose (125 mg/mL) | 0.721 | | 500 | NA |
| ADOV-15 | 0.25 | 0.25 | 2.5 | 0.1406 | 6 | | Sucrose (100 mg/mL) | 0.673 | | 100 | −100 |
| ADOV-16 | | | | | | | Sucrose (50 mg/mL) | 0.508 | | | |
| ADOV-17 | | | | | | | Mannitol (100 mg/mL) | 0.673 | | | |
| ADOV-18 | | | | | | | Mannitol (50 mg/mL) | 0.508 | | | |
| ADOV-19 | 0.5 | 0.5 | 5 | 0.2812 | — | 12 | Trehalose (125 mg/mL) | 0.576 | Citrate buffer | 200 | −100 |
| | | | | | | | | | (5 mM, pH 6.5) | | |
| ADOV-20 | 0.05 | 0.05 | 0.5 | 0.02812 | — | — | Trehalose (30 mg/mL) | 0.898 | Citrate Buffer | 1000 | −100 |
| | | | | | | | | | (10 mM, pH 6.5 | | |
| ADOV-21 | 0.25 | 0.25 | 2.5 | 0.1406 | — | — | Trehalose (30 mg/mL) | 0.838 | Citrate Buffer | 1000 | −100 |
| | | | | | | | | | (10 mM, pH 6.5 | | |

*Liquid formulation ADOV-14 was converted into dry powder via conventional shelf-freeze drying. The temperature of the formulation was slowly reduced from room temperature to −40° C. Then, the formulation was converted into dry powder using the same lyophilization cycle as other formulations that were processed using TFFD.

TABLE 2
Volume-based particle size distribution (i.e., Dᵥ(10), Dᵥ(50), and Dᵥ(90)), % T and span
values of AddaVax/OVA dry powder measured by Malvern Spraytec laser diffraction
system at 6 cm from the tip of the powder blower. Data obtained during the formation
phase, the fully-developed phase, and the dissipation phase are presented.

| Phase | Dᵥ(10) (μm) | Dᵥ(50) (μm) | Dᵥ(90) (μm) | Span | % T |
|---|---|---|---|---|---|
| The formation phase | 5.8 | 24.6 | 165.2 | 6.5 | 72.7 |
| The fully-developed phase | 6.3 | 26.1 | 168.2 | 6.2 | 75.7 |
| The dissipation phase | 6.8 | 33.4 | 193.9 | 5.6 | 85.8 |

All of the compositions and methods disclosed and claimed herein can be made and executed without undue experimentation in light of the present disclosure. While the compositions and methods of this disclosure have been described in terms of particular embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.

AboulFotouh et al., “Next-Generation COVID-19 Vaccines Should Take Efficiency of Distribution into Consideration,” AAPS PharmSciTech, 22, 126, 2021.

Alving et al., “Adjuvants for human vaccines,” Curr Opin Immunol, 24, 310-315, 2012.

Amorij et al., “Rational design of an influenza subunit vaccine powder with sugar glass technology: preventing conformational changes of haemagglutinin during freezing and freeze-drying,” Vaccine, 25, 6447-6457, 2007.

Chen et al., “Improving the reach of vaccines to low-resource regions, with a needle-free vaccine delivery device and long-term thermostabilization,” J Control Release, 152, 349-355, 2011.

Engstrom et al., “Formation of stable submicron protein particles by thin film freezing,” Pharm Res, 25, 1334-1346, 2008.

Heller et al., “Manipulation of Lyophilization-Induced Phase Separation: Implications For Pharmaceutical Proteins,” Biotechnology Progress, 13, 590-596, 1997.

Ko & Kang, “Immunology and efficacy of MF59-adjuvanted vaccines,” Hum Vaccin Immunother, 14, 3041-3045, 2018.

Randolph, “Phase separation of excipients during lyophilization: effects on protein stability,” J Pharm Sci, 86, 1198-1203, 1997.

van Diepen et al., “The adjuvant AlhydroGel elicits higher antibody titres than AddaVax when combined with HIV-1 subtype C gp140 from CAP256,” PLoS One, 13, 2018.

1. A dry oil-in-water (O/W) emulsion composition wherein said composition comprises a sugar or a sugar alcohol and an antigen.

2. The dry O/W composition of claim 1, wherein the antigen is an antigen in a subunit vaccine.

3. The dry O/W composition of claim 1, wherein said sugar or sugar alcohol is sucrose, trehalose, or mannitol.

4. The dry O/W composition of claim 1, wherein said dry O/W composition has a particle size distribution upon reconstitution within about 10-100% of the range of a corresponding liquid adjuvant composition.

5. The dry O/W composition of claim 1, wherein said sugar or a sugar alcohol is present at about 40-90 w/w.

6. The dry O/W composition of claim 1, wherein said antigen is influenza virus antigen, diphtheria and tetanus toxoids, hepatitis B surface antigen, major capsid protein of human papilloma virus, and severe acute respiratory syndrome coronavirus 2 (SARS-CoV-2) spike protein.

7. (canceled)

8. The dry O/W composition of claim 1, further comprising an excipient in addition to said sugar or sugar alcohol.

9. The dry O/W composition of claim 8, wherein said excipient is a salt, a buffer, a detergent, a polymer, an amino acid, a second sugar or sugar alcohol or a preservative.

10. The dry O/W composition of claim 9, wherein said excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, tri-n-butyl phosphate, ascorbic acid, cysteine hydrochloride, hydrochloric acid, hydrogen citrate, trisodium citrate, guanidine hydrochloride, mannitol, lactose, agarose, sorbitol, maltose, trehalose, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

11. The dry O/W composition of claim 8, comprising 0.3-27.5% w/w of said excipient.

12. The dry O/W composition of claim 1, wherein said dry O/W composition is prepared from a liquid vaccine.

13. The dry O/W composition of claim 1, wherein the oil component comprises squalene.

14. The dry O/W composition of claim 13, wherein said sugar is trehalose present at 40-90% w/w and the oil component is squalene.

15. (canceled)

16. A method for preparing an adjuvant thin film comprising:
applying a liquid adjuvant composition to a freezing surface, wherein said liquid adjuvant composition comprises an oil-in-water (O/W) nanoemulsion and a sugar or sugar alcohol;
allowing said liquid adjuvant composition to disperse and freeze on said freezing surface thereby forming a thin film.

17. The method of claim 16, wherein the adjuvant thin film further comprises an antigen, such as an antigen from a subunit vaccine.

18. The method of claim 16, wherein said sugar or sugar alcohol is sucrose, trehalose or mannitol.

19. The method of claim 16, wherein said adjuvant thin film has a particle size distribution upon reconstitution within 10-100% of the range of the liquid adjuvant composition.

20. The method of claim 16, wherein said sugar or sugar alcohol is present at 40-90 w/w.

21. The method of claim 17, wherein said antigen is an influenza virus antigen, diphtheria and tetanus toxoids, hepatitis B surface antigen, major capsid protein of human papilloma virus and severe acute respiratory syndrome coronavirus 2 (SARS-CoV-2) spike protein.

22. (canceled)

23. The method of claim 16, wherein said adjuvant thin film further comprises an excipient other than said sugar or sugar alcohol.

24-45. (canceled)
